(19) United States
(12) Patent Application Publication
(10) Pub. No.: US 2025/0172668 A1
(43) Pub. Date: May 29, 2025

(54) DETECTION DEVICE

(71) Applicant: SEIKO EPSON CORPORATION, Tokyo (JP)

(72) Inventors: Hiromu TAKAYAMA, Chino-shi (JP); Takefumi FUKAGAWA, Fujimi-machi (JP)

(73) Assignee: SEIKO EPSON CORPORATION, Tokyo (JP)

(21) Appl. No.: 18/962,362

(22) Filed: Nov. 27, 2024

(30) Foreign Application Priority Data
Nov. 29, 2023 (JP) 2023-201441

(51) Int. Cl. G01S 7/481

(52) U.S. Cl. CPC G01S 7/4813

(57) ABSTRACT

A detection device configured to be attached to a predetermined portion in a subject, including a base part including a surface orthogonal to a first direction, an acceleration detection part provided at the base part, a lid part configured to cover the acceleration detection and base parts, a control part provided at the lid part configured to detect a body movement of the subject based on an output of the acceleration detection part when the detection device is attached to the portion, a light emission part configured to emit light, a light reception part configured to receive light, and a housing part configured to house the base, acceleration detection, lid, control, light emission, and light reception parts. A height of a top surface of the light emission part in the first direction is equal to or greater than that of the bottom surface of the lid part.

The present application is based on, and claims priority from JP Application Serial Number 2023-201441, filed Nov. 29, 2023, the disclosure of which is hereby incorporated by reference herein in its entirety.

### BACKGROUND

### 1. Technical Field

This disclose relates to a detection device.

### 2. Related Art

Research and development are being conducted on detection devices that detect biological information such as pulse wave, pulse rate, and oxygen saturation in a non-invasive manner.

In view of this, there is known a detection device configured to be attached to a predetermined target portion of the subject person, including a light emission part that emits light, a light reception part that receives light, a biological information detection part that detects the biological information of the subject person based on the light reception amount at the light reception part of the light that is emitted from the light emission part and reflected inside the target portion, and an acceleration detection part that detects the acceleration of the target portion (see JP-A-2020-000596).

In a detection device such as that disclosed in JP-A-2020-000596, the biological information detection part and the acceleration detection part are disposed in respective different spaces. However, when reducing the size of the detection device, it is desirable to install the biological information detection part and the acceleration detection part in the same space. However, if the biological information detection part and the acceleration detection part are disposed in the same space, a part of light emitted from the biological information detection part may be absorbed by the acceleration detection part. In addition, the acceleration detection part is often dark gray. In this case, the acceleration detection part absorbs most of the light that is emitted from the biological information detection part to the acceleration detection part. Such light absorption at the acceleration detection part may lead to the thermal expansion of the acceleration detection part, and reduction in acceleration detection accuracy at the acceleration detection part, which is not desirable.

### SUMMARY

To solve the above-mentioned problems, a detection device according to an aspect of the present disclosure is configured to be attached to a predetermined portion in a subject person, the detection device including a base part including a surface orthogonal to a first direction, the first direction being a predetermined direction set as an upper direction, an acceleration detection part provided at the base part, a lid part configured to cover the acceleration detection part together with the base part on the base part, a control part provided at the lid part and configured to detect a body movement of the subject person based on an output of the acceleration detection part when the detection device is attached to the portion, a light emission part configured to emit light, a light reception part configured to receive light, and a housing part configured to house the base part, the acceleration detection part, the lid part, the control part, the light emission part, and the light reception part. A height of a top surface of the light emission part in the first direction is equal to or greater than the height of the bottom surface of the lid part in the first direction.

### BRIEF DESCRIPTION OF THE DRAWINGS

FIG. 1 is a front view illustrating an example of a configuration of a detection device 1.

FIG. 2 is a top view of the detection device 1 illustrated in FIG. 1.

FIG. 3 is a front view illustrating a first modification of the configuration of the detection device 1.

FIG. 4 is a top view of the detection device 1 illustrated in FIG. 3.

FIG. 5 is a front view illustrating a second modification of the configuration of the detection device 1.

FIG. 6 is a top view of the detection device 1 illustrated in FIG. 5.

FIG. 7 is a front view illustrating a third modification of the configuration of the detection device 1.

FIG. 8 is a top view of the detection device 1 illustrated in FIG. 7.

FIG. 9 is a front view illustrating a fourth modification of the configuration of the detection device 1.

FIG. 10 is a top view of the detection device 1 illustrated in FIG. 9.

FIG. 11 is a front view illustrating a fifth modification of the configuration of the detection device 1.

FIG. 12 is a top view of the detection device 1 illustrated in FIG. 11.

FIG. 13 is a front view illustrating a sixth modification of the configuration of the detection device 1.

FIG. 14 is a top view of the detection device 1 illustrated in FIG. 13.

FIG. 15 is a front view illustrating the seventh modification of the configuration of the detection device 1.

FIG. 16 is a top view of the detection device 1 illustrated in FIG. 15.

FIG. 17 is a front view illustrating the eighth modification of the configuration of the detection device 1.

FIG. 18 is a top view of the detection device 1 illustrated in FIG. 17.

FIG. 19 is a front view illustrating an example of a configuration of a detection device 2.

FIG. 20 is a top view of the detection device 2 illustrated in FIG. 19.

FIG. 21 is a front view illustrating a first modification of the configuration of the detection device 2.

FIG. 22 is a top view of the detection device 2 illustrated in FIG. 21.

FIG. 23 is a front view illustrating a second modification of the configuration of the detection device 2.

FIG. 24 is a top view of the detection device 2 illustrated in FIG. 23.

FIG. 25 is a front view illustrating a third modification of the configuration of the detection device 2.

FIG. 26 is a top view of the detection device 2 illustrated in FIG. 25.

FIG. 27 is a front view illustrating an example of the configuration of a detection device 3.

FIG. 28 is a top view of the detection device 3 illustrated in FIG. 27.

FIG. 29 is a front view illustrating a first modification of the configuration of the detection device 3.

FIG. 30 is a side view of the detection device 3 illustrated in FIG. 29.

FIG. 31 is a top view of the detection device 3 illustrated in FIG. 29.

FIG. 32 is a front view illustrating a second modification of the configuration of the detection device 3.

FIG. 33 is a front view illustrating a third modification of the configuration of the detection device 3.

### DESCRIPTION OF EMBODIMENTS

### Summary of Present Disclosure

The following describes an overview of the present disclosure. The present disclosure describes a detection device configured to be attached to a predetermined target portion of the subject person, including a light emission part that emits light, a light reception part that receives light, a biological information detection part that detects the biological information of the subject person based on the light reception amount at the light reception part of the light that is emitted from the light emission part and reflected inside the target portion, and an acceleration detection part that detects the acceleration of the target portion. In the case where the biological information detection part and the acceleration detection part are disposed in the same space, the size of the detection device can be reduced in comparison with the case where the biological information detection part and the acceleration detection part are disposed different respective spaces. However, the configuration in which the biological information detection part and the acceleration detection part are disposed in the same space may lead to the increase in the amount of the light that is emitted from the light emission part and enters the acceleration detection part without passing through the target portion, the amount of the light that is emitted from the light emission part and enters the light reception part as stray light, the amount of the light that is emitted from the light emission part and diffusely reflected in the space to enter the light reception part, and the like. The increase in the amount of the light that is emitted from the light emission part and enters the acceleration detection part without passing through the target portion may lead to the thermal expansion of the acceleration detection part, and reduction in acceleration detection accuracy at the acceleration detection part, which is not desirable. The increase in the amount of the light that is emitted from the light emission part and enters the light reception part as stray light may lead to reduction in detection accuracy of the biological information, which is not desirable. Further, the increase in the amount of the light that is emitted from the light emission part and diffusely reflected in the space to enter the light reception part may lead to reduction in detection accuracy of the biological information, which is not desirable. In view of this, in the present disclosure, configurations of a detection device that can solve at least one of the above-described problems are described. Such configurations are achieved by the following first to third embodiments, or combinations of some or all of the three embodiments, for example. In view of this, the following specifically describes the three embodiments. Note that the first to third embodiments, or combinations of some or all of the three embodiments may be combined with other configurations as long as the function of the detection device described below is not impaired.

### First Embodiment

A first embodiment is described below with reference to the accompanying drawings.

### Overview of Detection Device According To First Embodiment

First, an overview of a detection device according to the first embodiment is described.

The detection device according to the first embodiment is attached to a predetermined portion of a subject person. In the detection device, a predetermined first direction is set as an upward direction. In addition, the detection device includes a base part, an acceleration detection part, a lid part, a control part, a light emission part, a light reception part, and a housing part. The base part is a surface orthogonal to the first direction. The acceleration detection part is provided on the base part. The lid part covers the acceleration detection part together with the base part on the base part. The control part is provided on the lid part, and configured to detect the body movement based on the output of the acceleration detection part when the detection device is attached to the portion subject person. The light emission part emits light. The light reception part receives light. The housing part houses the base part, the acceleration detection part, the lid part, the control part, the light emission part and the light reception part. Further, the height of the top surface of the light emission part in the first direction is equal to or greater than the height of the bottom surface of the lid part in the first direction. In addition, the light emission part and the light reception part are provided on the same member. In this manner, the detection device can reduce the amount of light emitted from the light emission part to the acceleration detection part. As a result, the detection device can suppress the heating of the acceleration detection part due to incidence of light. Further, in the detection device, the light emission part and the light reception part that function as the biological information detection part, and the acceleration detection part are housed in the housing part. As a result, the detection device can be downsized in comparison with the case where the light emission part and the light reception part that function as the biological information detection part and the acceleration detection part are housed in separate housing parts. That is, the detection device can suppress the heating of the acceleration detection part due to incidence of light while reducing the size.

A configuration of the detection device according to the first embodiment is elaborated below.

### Configuration of Detection Device According To First Embodiment

A configuration of the detection device according to the first embodiment is described below with a detection device 1 as an example. In the first embodiment, the user of the detection device 1 is referred to as first user for convenience of description. In addition, in the first embodiment, viewing the detection device 1 toward a certain direction is referred to as viewing from that direction for convenience of description. Note that the first user is an example of the subject person.

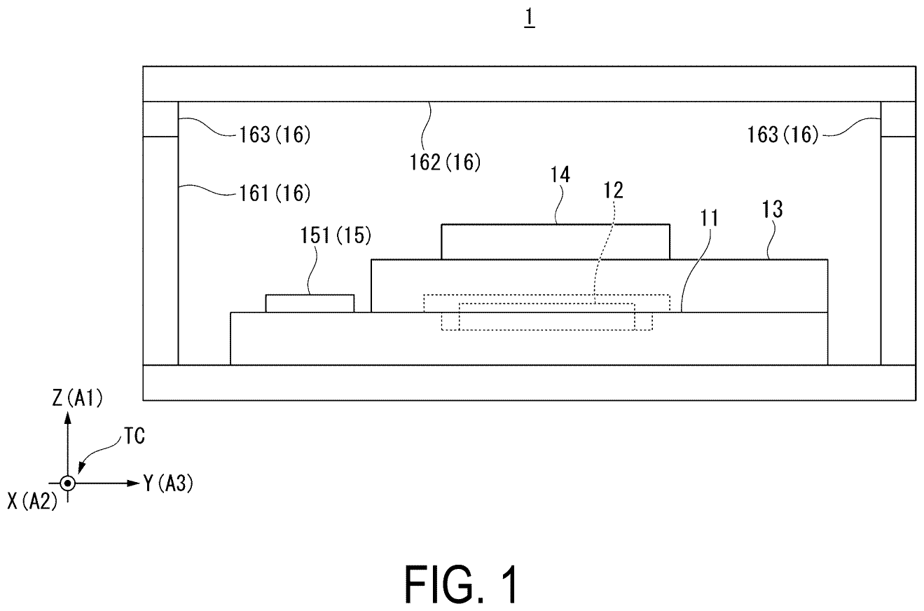

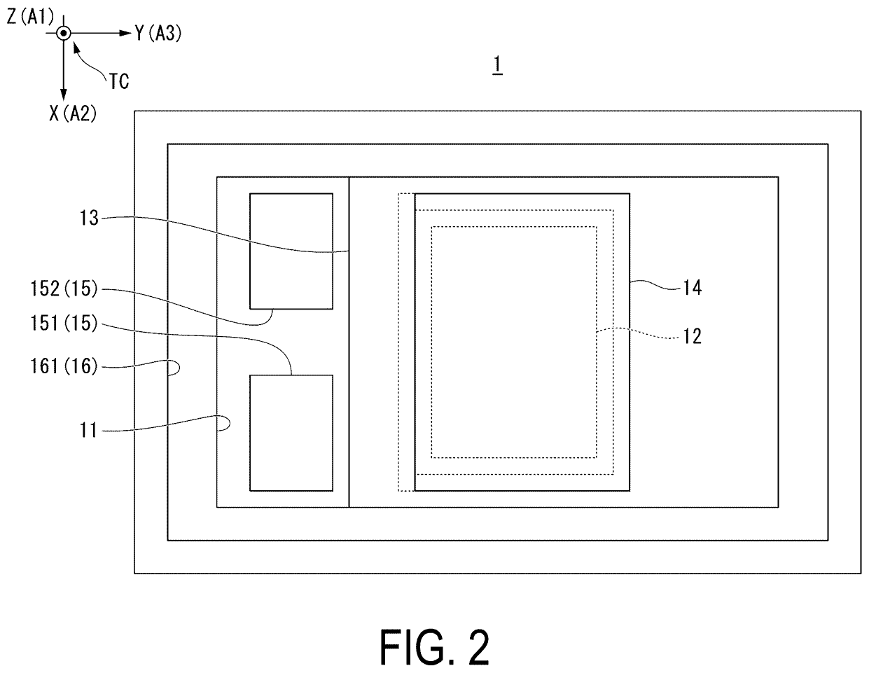

FIG. 1 is a front view illustrating an example of a configuration of the detection device 1. FIG. 2 is a top view of the detection device 1 illustrated in FIG. 1. Note that in FIG. 1, to clearly illustrate the inner configuration of the detection device 1, the member that covers the front surface of the inside of the detection device 1 is omitted. In addition, in FIG. 2, to clearly illustrate the inner configuration of the detection device 1, the member that covers the top surface of the inside of the detection device 1 is omitted. Here, a three-dimensional coordinate system TC represents the directions in the drawings in which the three-dimensional coordinate system TC is illustrated. In the present disclosure, for convenience of description, the X axis in the three-dimensional coordinate system TC is referred to simply as the X axis. In addition, in the present disclosure, for convenience of description, the Y axis in the three-dimensional coordinate system TC is simply referred to as the Y axis. In addition, in the present disclosure, for convenience of description, the Z axis in the three-dimensional coordinate system TC is simply referred to as the Z axis. In addition, in the present disclosure, for convenience of description, the positive direction in the Z axis is referred to as the upper side or upward direction, and the negative direction in the Z axis is referred to as the lower side or lower direction.

The detection device 1 is a device that detects biological information by using light. The first embodiment describes a case where the detection device 1 is a device that detects human biological information as an example. In this case, the detection device 1 detects pulse wave, pulse, oxygen saturation and the like as biological information, and is provided in vital devices such as smart watches, active trackers, smart rings, pulse oximeters, smart earphones, or the like, for example. Note that the detection device 1 may be configured to be provided in a wireless mouse, a door knob sensor, a sensor of a steering wheel of an automobile or the like. In the present disclosure, a case where the detection device 1 detects the biological information of the first user is described. In this case, the detection device 1 is attached to a predetermined portion of the first user. The portion is, but not limited to, the wrist or the like, for example. In the present disclosure, the portion is referred to as target portion. Note that the detection device 1 may be configured to detect the biological information of non-human animal, and configured to detect the biological information of plants.

More specifically, the detection device 1 is pressed against the skin of the target portion, and emits light in predetermined wavelength bands toward the skin. Then, the detection device 1 receives the reflection light of the light emitted toward the skin, and detects the pulse, oxygen saturation and the like on the basis of the temporal variation of the light reception amount of the received reflection light. Here, the material that reflects the light emitted by the detection device 1 is, but not limited to, hemoglobin in capillaries and the like, for example. In addition, for example, the detection device 1 uses light in the green wavelength band when detecting the pulse. The green wavelength band is 500 to 570 [nm]. In addition, for example, the detection device 1 uses light in the red wavelength band, light in the infrared wavelength band and the like when detecting the oxygen saturation. The red wavelength band is 630 to 680 [nm], and light in the infrared wavelength band is 850 to 1000 [nm].

The detection device 1 includes a base part 11, an acceleration detection part 12, a lid part 13, a control part 14, a biological information detection part 15, and a housing part 16, for example. Note that the detection device 1 also includes other members such as a transmission path, a connection terminal and the like for electrically coupling some or all of the base part 11, the acceleration detection part 12, the control part 14, and the biological information detection part 15. The transmission path is, but not limited to, a wire bonding, a conductive wire or the like, for example. However, in the present disclosure, the description for the other members is omitted. As such, in each drawing, illustration of the other members is omitted.

In the detection device 1, a predetermined direction A1 with respect to the base part 11 is set as an upward direction. The direction A1 may be any direction. In the present disclosure, a case where the direction A1 coincides with the Z axis positive direction is described. In view of this, in the present disclosure, the position of a certain surface in the direction A1 is described as the height of that surface. The direction A1 is an example of the first direction and the height direction.

The base part 11 is a substrate made of glass, for example. A semiconductor chip not illustrated in the drawing or the like is fixed to the surface of the base part 11 by a die attach material. Note that the base part 11 may be a substrate using phenol resin, polyimide resin, fluororesin, and epoxy resin, for example. The base part 11 includes a surface orthogonal to the direction A1. In the present disclosure, an example case where the base part 11 is a substrate having a shape of a rectangular flat plate with a top surface and a bottom surface orthogonal to the direction A1 as illustrated in FIGS. 1 and 2 is described. In addition, in the present disclosure, an example case where the long direction of the base part 11 is parallel to the Y axis, and the short direction of the base part 11 is parallel to the X axis as illustrated in FIGS. 1 and 2 is described. In addition, in the example illustrated in FIGS. 1 and 2, a recess in which the acceleration detection part 12 is provided is formed at the top surface of the base part 11. Note that the recess may not be formed at the top surface of the base part 11. In this case, the acceleration detection part 12 is provided at the top surface of the base part 11.

The acceleration detection part 12 is a sensor, device and the like for detecting the acceleration of the target portion in the case where the detection device 1 is attached to the target portion. The acceleration detection part 12 may be any sensor, device and the like as long as it is capable of detecting the acceleration. The acceleration detection part 12 outputs information representing the detected acceleration to the control part 14. In the example illustrated in FIGS. 1 and 2, the acceleration detection part 12 is composed of a silicon structure provided on the base part 11. The structure of the acceleration detection part 12 as a silicon structure may be a known structure, or a structure to be developed. As such, in the present disclosure, the description of the structure of the acceleration detection part 12 is omitted.

The acceleration detection part 12 is provided on the base part 11. More specifically, the acceleration detection part 12 is provided in the recess formed at the top surface of the base part 11. In the example illustrated in FIG. 1, the height of the acceleration detection part 12 in the direction A1 is greater than the depth of the recess in the direction A1. As such, in this example, in FIG. 1, the top surface of the acceleration detection part 12 is located on the upper side of the top surface of the base part 11.

The lid part 13 is an opaque member on the base part 11 that covers the acceleration detection part 12 together with the base part 11. In this manner, in the detection device 1, incidence of light past the lid part 13 on the acceleration detection part 12 is suppressed. The lid part 13 is a silicon cap, for example. In the example illustrated in FIGS. 1 and 2, a recess that covers the upper part of the acceleration detection part 12 is formed at the bottom surface of the lid part 13. Note that the lid part 13 may be other opaque members capable of covering the acceleration detection part 12 together with the base part 11 on the base part 11, instead of the silicon cap. The other member is, but not limited to, an opaque resin member or the like, for example.

The control part 14 is an IC (Integrated Circuit) chip that controls the acceleration detection part 12, the biological information detection part 15 and the like. The control part 14 is provided on the lid part 13. The control part 14 detects the body movement of the first user on the basis of the output of the acceleration detection part 12 in the case where the detection device 1 is attached to the target portion, for example. In the example illustrated in FIGS. 1 and 2, the control part 14 extends toward the direction parallel to the X axis. However, the control part 14 may be configured to extend toward a direction that is not parallel to the X axis.

The biological information detection part 15 includes a light emission part 151 and a light reception part 152.

The light emission part 151 is a light-emitting element, a light-emitting device or the like that emits light in predetermined wavelength bands. The light emission part 151 is controlled by the control part 14. The light emission part 151 is, for example, an LED (Light Emitting Diode), but may alternatively be other light-emitting devices or other light-emitting elements such as an OLED (Organic Light Emitting Diode), a μ (Micro) LED, and a VCSEL (Vertical Cavity Surface Emitting Laser). The light emission part 151 emits light toward the target portion in the case where the detection device 1 is attached to the target portion. The wavelength band is, but not limited to, the above-described green wavelength band, red wavelength band, infrared wavelength band and the like, for example. Note that the depth of entry of light emitted from the light emission part 151 into the inside of the target portion differs depending on the wavelength band, intensity and the like of the light emitted from the light emission part 151.

Specifically, the detection device 1 can accurately detect the desired biological information by adjusting at least one of the wavelength band, intensity and the like of the light emitted from the light emission part 151.

In the example illustrated in FIGS. 1 and 2, the light emission part 151 is provided on the base part 11. In addition, in this example, the height of the top surface of the light emission part 151 is greater than the height of the bottom surface of the lid part 13. Here, the top surface of the light emission part 151 is the uppermost surface among the surfaces of the light emission part 151. Note that in the case where the uppermost surface among the surfaces of the light emission part 151 and the light-emitting surface of the light emission part 151 that emits light do not coincide with each other, the top surface of the light emission part 151 is the light-emitting surface. In the detection device 1, in the case where the height of the top surface of the light emission part 151 is greater than the height of the bottom surface of the lid part 13, light emitted from the light emission part 151 does not travel in the direction toward the base part 11 unless it is reflected. Further, as described above, the lid part 13 is an opaque member. Thus, in this case, in the detection device 1, the amount of light emitted from the light emission part 151 to the acceleration detection part 12 can be reduced. As a result, the detection device 1 can suppress the heating of the acceleration detection part 12 due to incidence of light. Note that the height of the top surface of the light emission part 151 may be the same as the height of the bottom surface of the lid part 13. Even in this case, in the detection device 1, the amount of light emitted from the light emission part 151 to the acceleration detection part 12 can be reduced. In addition, the light emission part 151 may be configured to be provided on the base part 11 with a member such as a spacer therebetween.

The light reception part 152 is a light-receiving element, a light reception device or the like that receives light. In the case where the detection device 1 is attached to the target portion, the light reception part 152 receives at least a part of the light that is emitted from the light emission part 151 and reflected inside the target portion. The light reception part 152 outputs information representing the amount of received light to the control part 14 as information representing the intensity of the received light.

Here, the above-described control part 14 detects the biological information of the first user in accordance with the intensity of the light received by the light reception part 152. In this detection of biological information, the control part 14 corrects the biological information in accordance with the body movement of the first user detected in accordance with the output of the acceleration detection part 12. This correction of biological information means, but not limited to, stop of the detection of biological information in the case where the body movement of the first user is a body movement of the predetermined type, removal, from the biological information, of the noise that is estimated to be generated in the case where the body movement of the first user is a body movement of the predetermined type, and the like. In addition, stop of the detection of biological information in the case where the body movement of the first user is a body movement of the predetermined type is achieved by a method of stopping the acquisition of the information output from the light reception part 152 by the control part 14, a method of stopping light emission at the light emission part 151 and the like, but may be achieved by other methods.

In the example illustrated in FIGS. 1 and 2, the light reception part 152 is provided on the base part 11 together with the light emission part 151. In addition, in this example, the height of the top surface of the light reception part 152 is the same as the height of the top surface of the light emission part 151. Note that the height of the top surface of the light reception part 152 may be different from the height of the top surface of the light emission part 151. In FIG. 1, the light reception part 152 is hidden behind the light emission part 151. In addition, the light reception part 152 may be configured to be provided on the base part 11 with a member such as a spacer therebetween. In the case where the height of the top surface of the light reception part 152 is different from the height of the top surface of the light emission part 151, the detection device 1 can suppress a situation where at least a part of the light that is emitted from the light emission part 151 and travels toward the light reception part 152 directly impinges on the light reception part 152 so as to be received by the light reception part 152. For example, in the case where the top surface of the light emission part 151 is higher than the top surface of the light reception part 152, most of the light emitted from the light emission part 151 travels upward without travelling toward the light reception part 152. On the other hand, for example, in the case where the top surface of the light emission part 151 is lower than the top surface of the light reception part 152, at least a part of the light that is emitted from the light emission part 151 and travels toward the light reception part 152 is reflected at the side surface of the light reception part 152, and does not reach the light-receiving element provided in the light reception part 152. Thus, in this case, the detection device 1 can suppress a situation where at least a part of the light that is emitted from the light emission part 151 and travels toward the light reception part 152 directly impinges on the light reception part 152 so as to be received by the light reception part 152. In the detection device 1, the difference in height of the top surfaces of the light emission part 151 and the light reception part 152 provided on the same member may be provided by using a spacer or the like, or by using the light emission part 151 and the light reception part 152 with different heights.

In addition, in the example illustrated in FIGS. 1 and 2, the light reception part 152 is provided side by side with the light emission part 151 on the base part 11 in a direction A2 that intersects the direction A1. In the present disclosure, as an example, a case where the direction A2 coincides with the positive direction of the X axis as illustrated in FIGS. 1 and 2 is described. In this case, the above-described control part 14 extends in the direction A2 in this example. The direction A2 is an example of each of the second direction and the arrangement direction.

In addition, in the example illustrated in FIGS. 1 and 2, the light reception part 152 is provided side by side with the control part 14 on the base part 11 in a direction A3 that intersects the direction A1 and the direction A2. In the present disclosure, as an example, a case where the direction A3 coincides with the positive direction of the Y axis as illustrated in FIGS. 1 and 2 is described. The direction A3 is an example of each of the third direction and the width direction.

Note that further, the biological information detection part 15 may be configured to include a processor not illustrated in the drawing that detects the biological information of the first user on the basis of the temporal variation of the amount of light received by the light reception part 152. When this processor detects the biological information, the processor outputs the detected biological information to the control part 14. Specifically, in this case, the control part 14 does not acquire the information output from the light reception part 152, but acquires the biological information output from the processor. That is, in this case, the control part 14 does not perform detection of biological information. As such, the processor is communicatively coupled with the light reception part 152 and the control part 14.

The housing part 16 houses the base part 11, the acceleration detection part 12, the lid part 13, the control part 14, and the biological information detection part 15. The housing part 16 is a container with a cuboid shape as a whole, for example. In this case, the housing part 16 is composed of a first member 161 and a second member 162.

The first member 161 is a member provided with a recess with an upper opening, and houses the base part 11, the acceleration detection part 12, the lid part 13, the control part 14, and the biological information detection part 15. In the side walls of the first member 161, the side wall on the positive direction side in the X axis is the member omitted in FIG. 1 that covers the front surface of the inside of the detection device 1.

The second member 162 is a rectangular plate-shaped member made of glass that closes the recess of the first member 161 from the upper side. The second member 162 is bonded to the first member 161 with an adhesive 163. The second member 162 may be configured to be assembled to the first member 161 without using the adhesive 163. In addition, the second member 162 is a member that makes contact with the skin as the target portion when the detection device 1 is attached to the target portion. In addition, the second member 162 is a member omitted in FIG. 2 as the member that covers the top surface of the inside of the detection device 1. In addition, the adhesive 163 is also omitted in FIG. 2 as with the second member 162. Note that for example, in the case where the inside of the recess of the first member 161 is filled with transparent resin, the housing part 16 may not include the second member 162. That is, the housing part 16 may be composed of the first member 161 and the resin.

The housing part 16 composed of the first member 161 and the second member 162 houses the biological information detection part 15 together with the acceleration detection part 12 as described above. In this manner, the detection device 1 can be downsized in comparison with the case where the biological information detection part 15 and the acceleration detection part 12 are housed in separate housing parts.

As described above, in the detection device 1 illustrated in FIGS. 1 and 2, the height of the top surface of the light emission part 151 is equal to or greater than the height of the bottom surface of the lid part 13. In this manner, the detection device 1 can reduce the amount of light emitted from the light emission part 151 to the acceleration detection part 12. As a result, the detection device 1 can suppress the heating of the acceleration detection part 12 due to incidence of light. In addition, in the detection device 1 illustrated in FIGS. 1 and 2, the acceleration detection part 12 and the biological information detection part 15 are housed in the housing part 16. In this manner, the detection device 1 can be downsized in comparison with the case where the biological information detection part 15 and the acceleration detection part 12 are housed in separate housing parts. That is, the detection device 1 can suppress the heating of the acceleration detection part 12 due to incidence of light while reducing the size.

Note that in the first embodiment, the height of the top surface of the light emission part 151 and the height of the top surface of the light reception part 152 are both lower than the height of the top surface of the control part 14. In this case, the thickness of the detection device 1 in the direction A1 can be reduced, and thus the detection device 1 can be downsized as a whole. To obtain this effect, the height of the top surface of the light emission part 151 and the height of the top surface of the light reception part 152 may both be the same as the height of the top surface of the control part 14. In the detection device 1, in the case where this effect does not have to be achieved, at least one of the height of the top surface of the light emission part 151 and the height of the top surface of the light reception part 152 may be higher than the height of the top surface of the control part 14.

### First Modification of First Embodiment

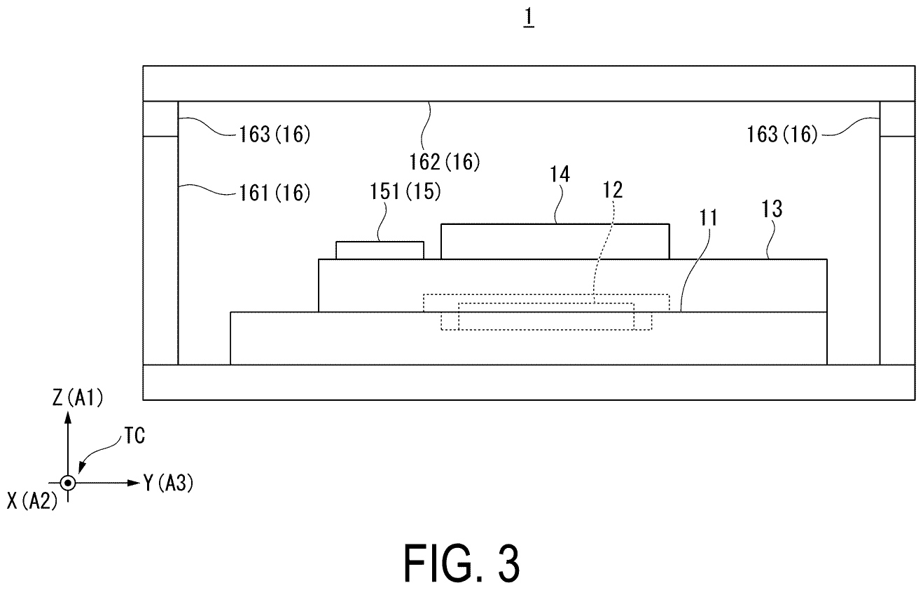

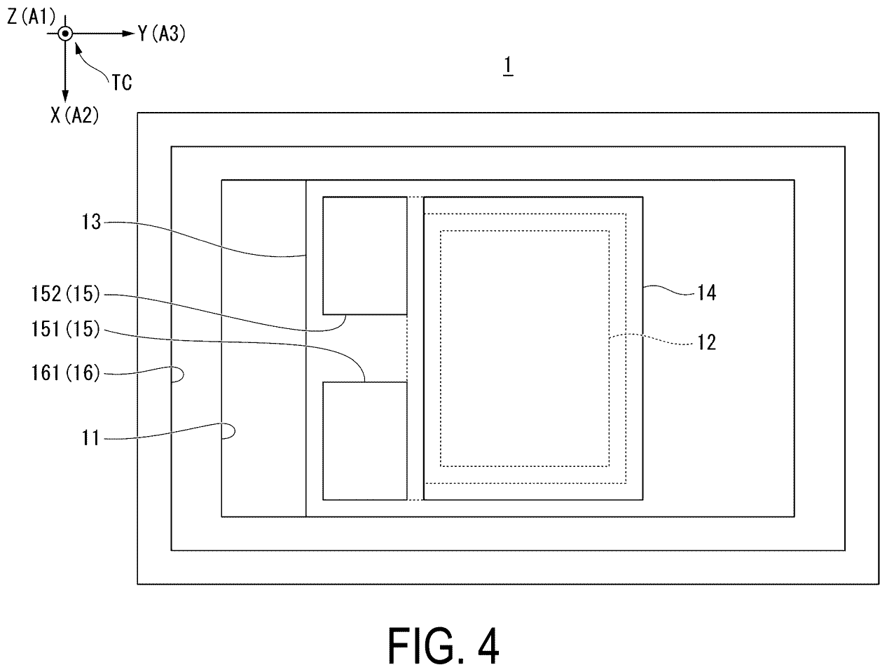

In a first modification of the first embodiment, the light emission part 151 and the light reception part 152 are both provided on the lid part 13. FIG. 3 is a front view illustrating the first modification of the configuration of the detection device 1. FIG. 4 is a top view of the detection device 1 illustrated in FIG. 3. Note that in FIG. 3, to clearly illustrate the inner configuration of the detection device 1, the member that covers the front surface of the inside of the detection device 1 is omitted. In addition, in FIG. 4, to clearly illustrate the inner configuration of the detection device 1, the member that covers the top surface of the inside of the detection device 1 is omitted.

In the example illustrated in FIGS. 3 and 4, the light emission part 151 is provided on the lid part 13. As such, also in this example, the height of the top surface of the light emission part 151 is greater than the height of the bottom surface of the lid part 13. Therefore, also in this case, in the detection device 1, the amount of light emitted from the light emission part 151 to the acceleration detection part 12 can be reduced. As a result, the detection device 1 can suppress the heating of the acceleration detection part 12 due to incidence of light. Note that the light emission part 151 may be provided on the lid part 13 with a member such as a spacer therebetween.

In addition, in the example illustrated in FIGS. 3 and 4, the light reception part 152 is provided on the lid part 13 together with the light emission part 151. In addition, also in this example, the height of the top surface of the light reception part 152 is the same as the height of the top surface of the light emission part 151. Note that the height of the top surface of the light reception part 152 may be different from the height of the top surface of the light emission part 151. In FIG. 3, the light reception part 152 is hidden behind the light emission part 151. Note that the light reception part 152 may be provided on the lid part 13 with a member such as a spacer therebetween.

In addition, in the example illustrated in FIGS. 3 and 4, the light reception part 152 is provided side by side with the light emission part 151 in the direction A2 on the lid part 13.

In addition, also in the example illustrated in FIGS. 3 and 4, the light reception part 152 is provided side by side with the control part 14 in the direction A3 on the lid part 13.

As described above, also in the detection device 1 illustrated in FIGS. 3 and 4, the height of the top surface of the light emission part 151 is equal to or greater than the height of the bottom surface of the lid part 13. In this manner, the detection device 1 can reduce the amount of light emitted from the light emission part 151 to the acceleration detection part 12. As a result, the detection device 1 can suppress the heating of the acceleration detection part 12 due to incidence of light. In addition, also in the detection device 1 illustrated in FIGS. 3 and 4, the acceleration detection part 12 and the biological information detection part 15 are housed in the housing part 16. In this manner, the detection device 1 can be downsized in comparison with the case where the biological information detection part 15 and the acceleration detection part 12 are housed in separate housing parts. That is, the detection device 1 can suppress the heating of the acceleration detection part 12 due to incidence of light while reducing the size.

Note that also in the first modification of the first embodiment, the height of the top surface of the light emission part 151 and the height of the top surface of the light reception part 152 are both lower than the height of the top surface of the control part 14. In this case, the thickness of the detection device 1 in the direction A1 can be reduced, and thus the detection device 1 can be downsized as a whole. To obtain this effect, the height of the top surface of the light emission part 151 and the height of the top surface of the light reception part 152 may both be the same as the height of the top surface of the control part 14. In the detection device 1, in the case where this effect does not have to be achieved, at least one of the height of the top surface of the light emission part 151 and the height of the top surface of the light reception part 152 may be higher than the height of the top surface of the control part 14.

### Second Modification of First Embodiment

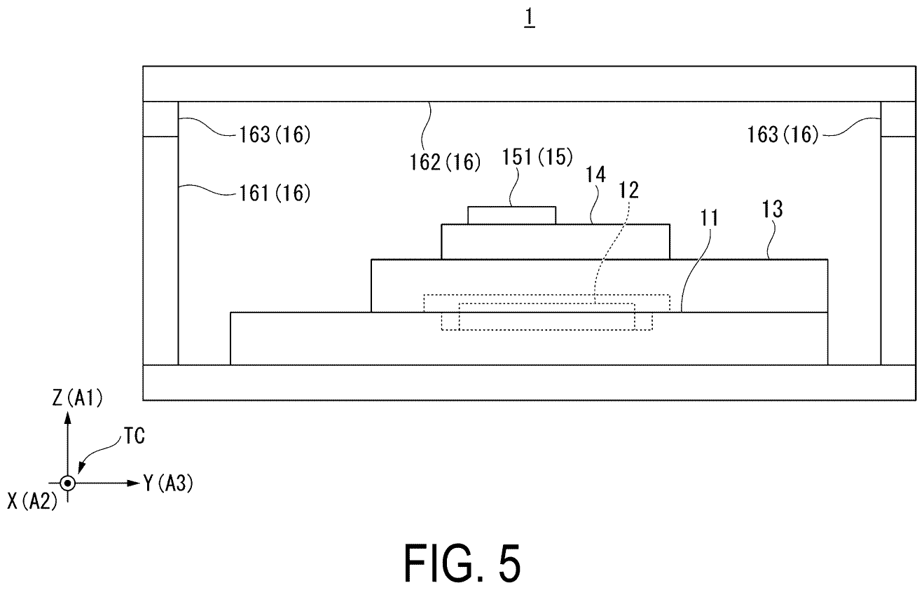

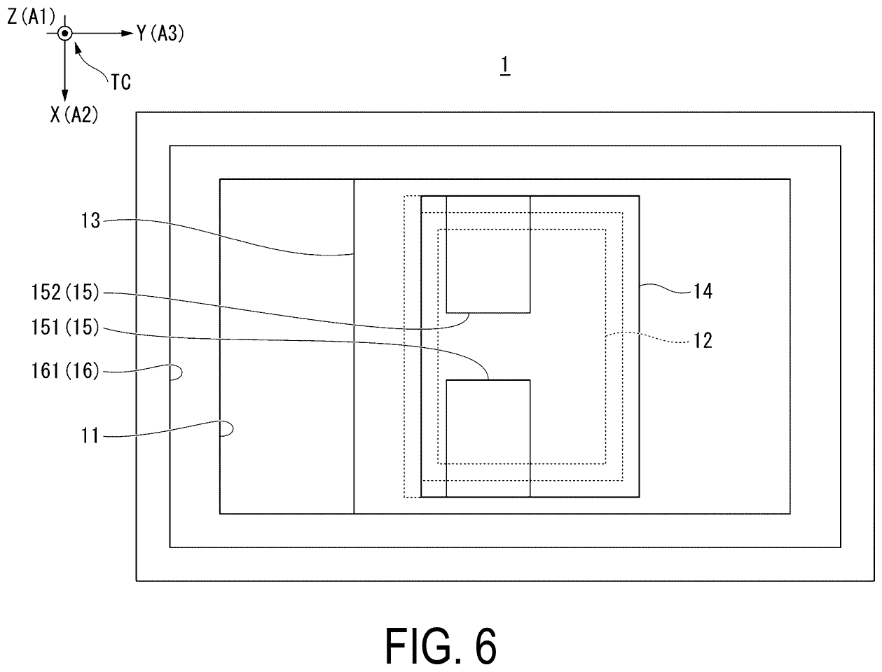

In the second modification of the first embodiment, the light emission part 151 and the light reception part 152 are both provided on the control part 14. FIG. 5 is a front view illustrating a second modification of the configuration of the detection device 1. FIG. 6 is a top view of the detection device 1 illustrated in FIG. 5. Note that in FIG. 5, to clearly illustrate the inner configuration of the detection device 1, the member that covers the front surface of the inside of the detection device 1 is omitted. In addition, in FIG. 6, to clearly illustrate the inner configuration of the detection device 1, the member that covers the top surface of the inside of the detection device 1 is omitted.

In the example illustrated in FIGS. 5 and 6, the light emission part 151 is provided on the control part 14. As such, also in this example, the height of the top surface of the light emission part 151 is greater than the height of the bottom surface of the lid part 13. Therefore, also in this case, in the detection device 1, the amount of light emitted from the light emission part 151 to the acceleration detection part 12 can be reduced. As a result, the detection device 1 can suppress the heating of the acceleration detection part 12 due to incidence of light. Note that the light emission part 151 may be provided on the control part 14 with a member such as a spacer therebetween.

In addition, in the example illustrated in FIGS. 5 and 6, the light reception part 152 is provided on the control part 14 together with the light emission part 151. In addition, also in this example, the height of the top surface of the light reception part 152 is the same as the height of the top surface of the light emission part 151. Note that the height of the top surface of the light reception part 152 may be different from the height of the top surface of the light emission part 151. In FIG. 5, the light reception part 152 is hidden behind the light emission part 151. Note that the light reception part 152 may be provided on the control part 14 with a member such as a spacer therebetween.

In addition, in the example illustrated in FIGS. 5 and 6, the light reception part 152 is provided side by side with the light emission part 151 in the direction A2 on the lid part 13.

As described above, also in the detection device 1 illustrated in FIGS. 5 and 6, the height of the top surface of the light emission part 151 is equal to or greater than the height of the bottom surface of the lid part 13. In this manner, the detection device 1 can reduce the amount of light emitted from the light emission part 151 to the acceleration detection part 12. As a result, the detection device 1 can suppress the heating of the acceleration detection part 12 due to incidence of light. In addition, also in the detection device 1 illustrated in FIGS. 5 and 6, the acceleration detection part 12 and the biological information detection part 15 are housed in the housing part 16. In this manner, the detection device 1 can be downsized in comparison with the case where the biological information detection part 15 and the acceleration detection part 12 are housed in separate housing parts. That is, the detection device 1 can suppress the heating of the acceleration detection part 12 due to incidence of light while reducing the size.

### Third Modification of First Embodiment

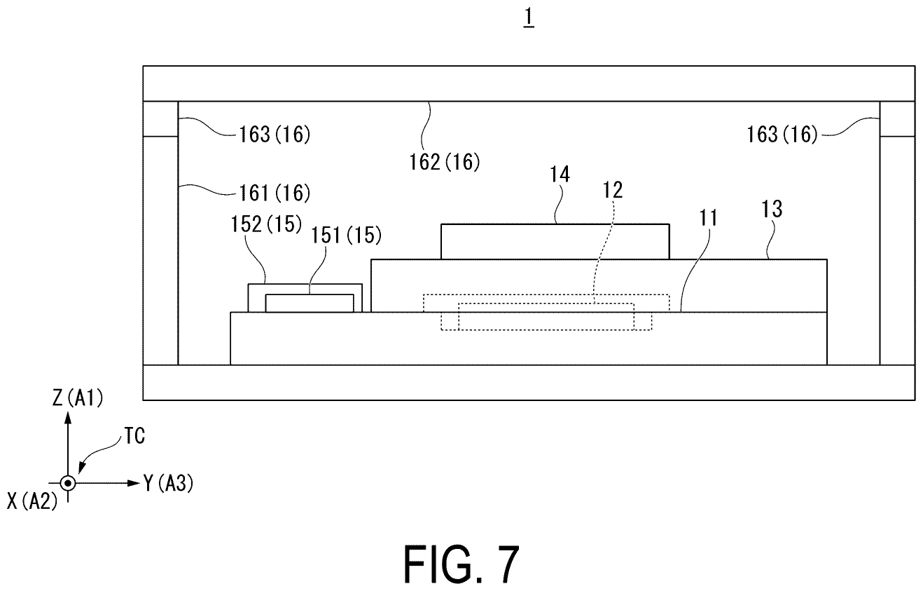

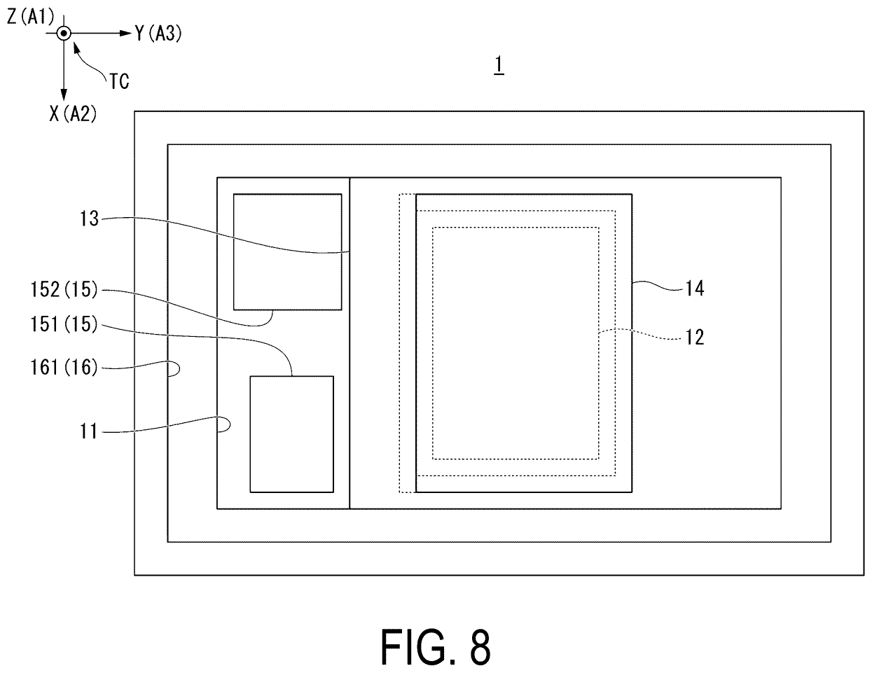

In a third modification of the first embodiment, the size of the light emission part 151 is different from the size of the light reception part 152. FIG. 7 is a front view illustrating a third modification of the configuration of the detection device 1. FIG. 8 is a top view of the detection device 1 illustrated in FIG. 7. Note that in FIG. 7, to clearly illustrate the inner configuration of the detection device 1, the member that covers the front surface of the inside of the detection device 1 is omitted. In addition, in FIG. 8, to clearly illustrate the inner configuration of the detection device 1, the member that covers the top surface of the inside of the detection device 1 is omitted.

In the example illustrated in FIGS. 7 and 8, the light emission part 151 and the light reception part 152 are both provided on the base part 11 as in the example illustrated in FIGS. 1 and 2. However, unlike in the first embodiment, in FIG. 7, a part of the light reception part 152 located behind the light emission part 151 is visible. The reason for this is that as viewed toward the direction A2, the size of the light reception part 152 is greater than the size of the light emission part 151. In addition, FIG. 8 shows that as viewed toward the direction opposite to the direction A1, the size of the light reception part 152 is also greater than the size of the light emission part 151. Note that the size relationship between the light emission part 151 and the light reception part 152 may be reversed.

Here, also in the example illustrated in FIGS. 7 and 8, the height of the top surface of the light emission part 151 is greater than the height of the bottom surface of the lid part 13. Therefore, also in this case, in the detection device 1, the amount of light emitted from the light emission part 151 to the acceleration detection part 12 can be reduced.

Specifically, the detection device 1 can suppress the heating of the acceleration detection part 12 due to incidence of light regardless of the size relationship between the light emission part 151 and the light reception part 152 as long as the height of the top surface of the light emission part 151 is greater than the height of the bottom surface of the lid part 13. Note that in the example illustrated in FIGS. 7 and 8, both the light emission part 151 and the light reception part 152 may be provided on the lid part 13, not on the base part 11, or may be provided on the control part 14.

As described above, also in the detection device 1 illustrated in FIGS. 7 and 8, the height of the top surface of the light emission part 151 is equal to or greater than the height of the bottom surface of the lid part 13. In this manner, the detection device 1 can reduce the amount of light emitted from the light emission part 151 to the acceleration detection part 12. As a result, the detection device 1 can suppress the heating of the acceleration detection part 12 due to incidence of light. In addition, also in the detection device 1 illustrated in FIGS. 7 and 8, the acceleration detection part 12 and the biological information detection part 15 are housed in the housing part 16. In this manner, the detection device 1 can be downsized in comparison with the case where the biological information detection part 15 and the acceleration detection part 12 are housed in separate housing parts. That is, the detection device 1 can suppress the heating of the acceleration detection part 12 due to incidence of light while reducing the size.

### Fourth Modification of First Embodiment

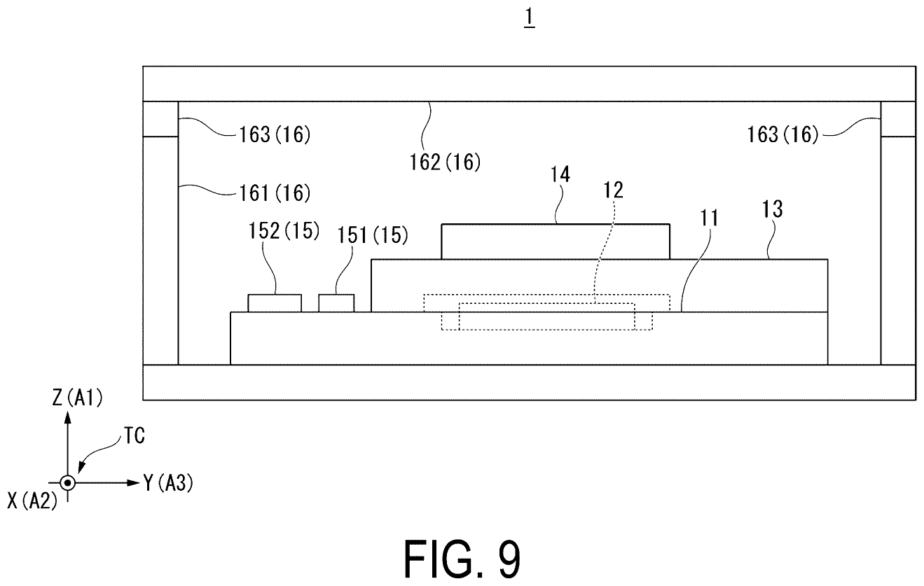

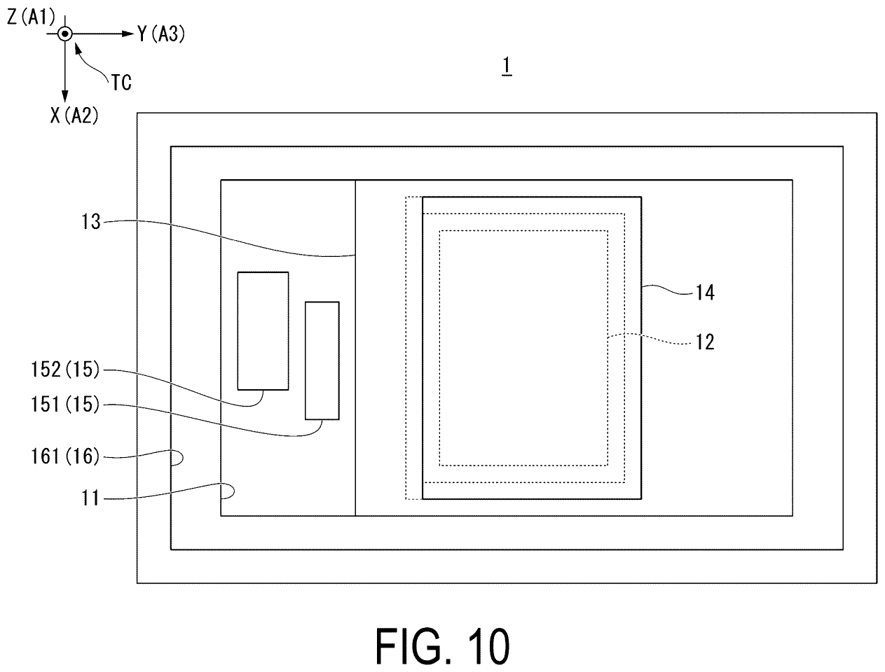

In a fourth modification of the first embodiment, the light emission part 151 is disposed side by side with the light reception part 152 in the direction A3. FIG. 9 is a front view illustrating a fourth modification of the configuration of the detection device 1. FIG. 10 is a top view of the detection device 1 illustrated in FIG. 9. Note that in FIG. 9, to clearly illustrate the inner configuration of the detection device 1, the member that covers the front surface of the inside of the detection device 1 is omitted. In addition, in FIG. 10, to clearly illustrate the inner configuration of the detection device 1, the member that covers the top surface of the inside of the detection device 1 is omitted.

In the example illustrated in FIGS. 9 and 10, the light emission part 151 and the light reception part 152 are both provided on the base part 11 as in the example illustrated in FIGS. 1 and 2. However, unlike in the first embodiment, in FIGS. 9 and 10, the light emission part 151 is not disposed side by side with the light reception part 152 in the direction A2 but is disposed side by side with the light reception part 152 in the direction A3. As such, as viewed toward the direction A2, in the detection device 1, the light emission part 151, the light reception part 152 and the control part 14 are arranged in the order of the light reception part 152, the light emission part 151, and the control part 14 in the direction A3.

Note that the light emission part 151 may be disposed side by side with the light reception part 152 in a direction different from the direction A2 and the direction A3 on the base part 11.

In addition, the light emission part 151 and the light reception part 152 may both be provided on the lid part 13 and disposed side by side in the direction A2. In addition, the light emission part 151 and the light reception part 152 may both be provided on the lid part 13, and disposed side by side in a direction different from the direction A2 and the direction A3 on the lid part 13.

In addition, the light emission part 151 and the light reception part 152 may be both provided on the control part 14, and disposed side by side in the direction A2. In addition, the light emission part 151 and the light reception part 152 may be both provided on the control part 14, and disposed side by side in a direction different from the direction A2 and the direction A3 on the control part 14.

Here, also in the example illustrated in FIGS. 9 and 10, the height of the top surface of the light emission part 151 is greater than the height of the bottom surface of the lid part 13. Therefore, also in this case, in the detection device 1, the amount of light emitted from the light emission part 151 to the acceleration detection part 12 can be reduced. Specifically, as long as the height of the top surface of the light emission part 151 is greater than the height of the bottom surface of the lid part 13, the detection device 1 can suppress the heating of the acceleration detection part 12 due to incidence of light regardless of the direction in which the light emission part 151 and the light reception part 152 are arranged on any of the base part 11, the lid part 13, and the control part 14.

As described above, also in the detection device 1 illustrated in FIGS. 9 and 10, the height of the top surface of the light emission part 151 is equal to or greater than the height of the bottom surface of the lid part 13. In this manner, the detection device 1 can reduce the amount of light emitted from the light emission part 151 to the acceleration detection part 12. As a result, the detection device 1 can suppress the heating of the acceleration detection part 12 due to incidence of light. In addition, also in the detection device 1 illustrated in FIGS. 9 and 10, the acceleration detection part 12 and the biological information detection part 15 are housed in the housing part 16. In this manner, the detection device 1 can be downsized in comparison with the case where the biological information detection part 15 and the acceleration detection part 12 are housed in separate housing parts. That is, the detection device 1 can suppress the heating of the acceleration detection part 12 due to incidence of light while reducing the size.

### Fifth Modification of First Embodiment

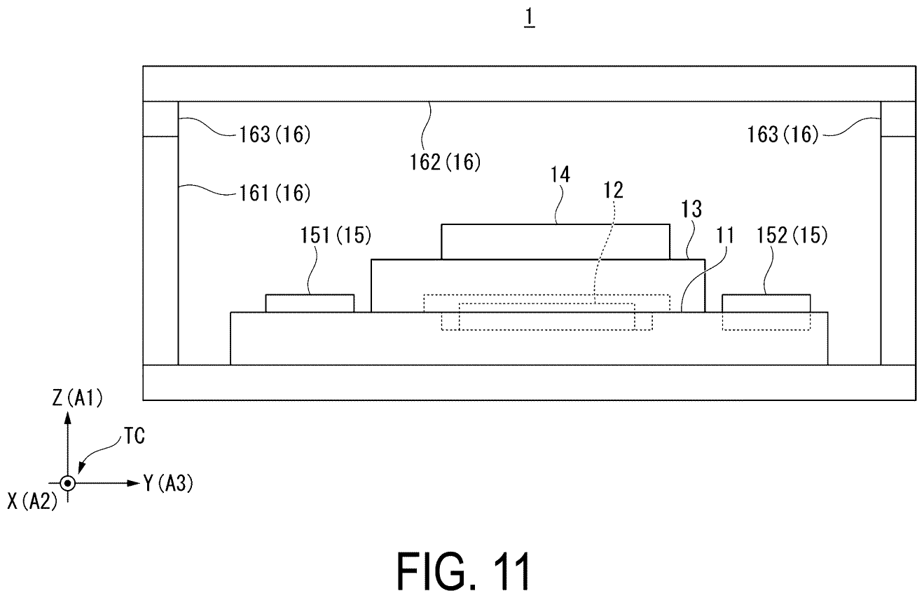

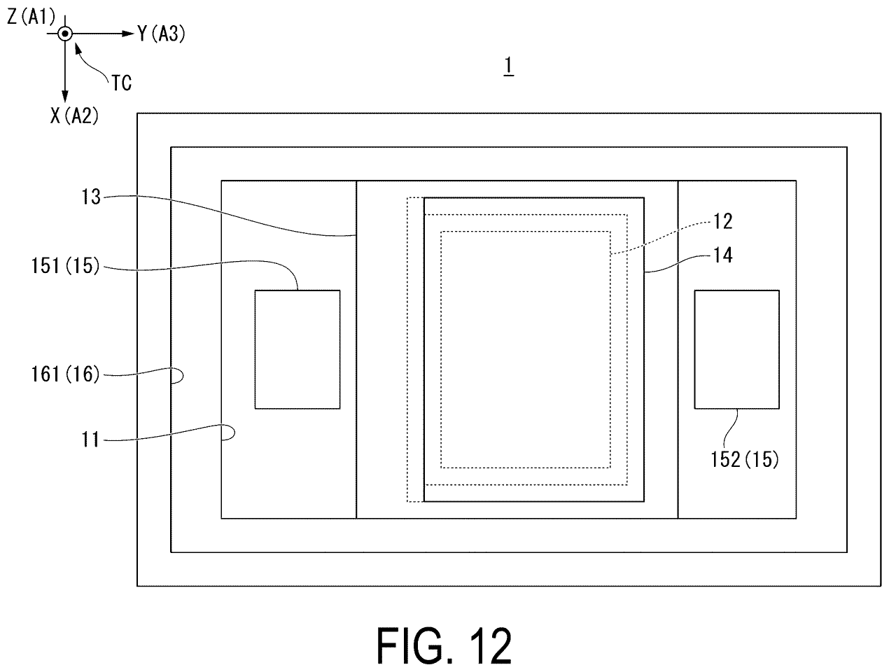

In a fifth modification of the first embodiment, the light emission part 151 and the light reception part 152 are both provided on the base part 11, the light emission part 151 is disposed side by side with the light reception part 152 and the control part 14 in the direction A3, and the control part 14 is located between the light emission part 151 and the light reception part 152 in the direction A3. FIG. 11 is a front view illustrating a fifth modification of the configuration of the detection device 1. FIG. 12 is a top view of the detection device 1 illustrated in FIG. 11. Note that in FIG. 11, to clearly illustrate the inner configuration of the detection device 1, the member that covers the front surface of the inside of the detection device 1 is omitted. In addition, in FIG. 12, to clearly illustrate the inner configuration of the detection device 1, the member that covers the top surface of the inside of the detection device 1 is omitted.

In the example illustrated in FIGS. 11 and 12, the light emission part 151 and the light reception part 152 are both provided on the base part 11 as in the example illustrated in FIGS. 1 and 2. However, unlike in the first embodiment, in FIGS. 11 and 12, the light emission part 151 is not disposed side by side with the light reception part 152 in the direction A2, but is disposed side by side with the control part 14 and the light reception part 152 in the order of the light emission part 151, the control part 14, and the light reception part 152 in the direction A3. Specifically, in this example, the control part 14 is located between the light emission part 151 and the light reception part 152 in the direction A3.

Here, also in the example illustrated in FIGS. 11 and 12, the height of the top surface of the light emission part 151 is greater than the height of the bottom surface of the lid part 13. Therefore, also in this case, in the detection device 1, the amount of light emitted from the light emission part 151 to the acceleration detection part 12 can be reduced. Specifically, as long as the height of the top surface of the light emission part 151 is greater than the height of the bottom surface of the lid part 13, the detection device 1 can suppress the heating of the acceleration detection part 12 due to incidence of light even when the light emission part 151 and the light reception part 152 are disposed side by side with the control part 14 therebetween on the base part 11.

In addition, in order to achieve size reduction of the detection device 1, it is desirable that the biological information detection part 15 and the acceleration detection part 12 be disposed in the same housing part 16 as in the detection device 1, and that the light emission part 151 and the light reception part 152 be close to each other. However, if the light emission part 151 and the light reception part 152 are close to each other in the housing part 16 in which the biological information detection part 15 and the acceleration detection part 12 are both disposed, a part of the light emitted from the light emission part 151 is likely to be received by the light reception part 152 as stray light without passing through the inside of the target portion. This leads to reduction in the detection accuracy of the biological information by the detection device 1, which is not desirable. Note that a method of solving this problem by disposing a light blocking member between the light emission part 151 and the light reception part 152 may hinder the size reduction of the detection device 1, and therefore cannot be employed in many cases.

However, this problem is solved in the detection device 1 by providing the light emission part 151 and the light reception part 152 with the control part 14 therebetween on the base part 11 as in the detection device 1 illustrated in FIGS. 11 and 12. The reason for this is that in the example illustrated in FIGS. 11 and 12, the light that is emitted from the light emission part 151 and travels toward the light reception part 152 is reflected by the lid part 13 and the control part 14, and is less likely to be received by the light reception part 152 as stray light. That is, with the control part 14 provided between the light emission part 151 and the light reception part 152 in the direction in which the light emission part 151 and the light reception part 152 are arranged in the housing part 16, the detection device 1 can reduce the amount of the stray light incident on the light reception part 152 while achieving size reception. In addition, the detection device 1 can reduce the amount of the stray light incident on the light reception part 152 in this manner, and therefore does not need to include a light blocking member for blocking light to prevent stray light incident on the light reception part 152. As a result, the detection device 1 can be more reliably downsized.

As described above, also in the detection device 1 illustrated in FIGS. 11 and 12, the height of the top surface of the light emission part 151 is equal to or greater than the height of the bottom surface of the lid part 13. In this manner, the detection device 1 can reduce the amount of light emitted from the light emission part 151 to the acceleration detection part 12. As a result, the detection device 1 can suppress the heating of the acceleration detection part 12 due to incidence of light. In addition, also in the detection device 1 illustrated in FIGS. 11 and 12, the acceleration detection part 12 and the biological information detection part 15 are housed in the housing part 16. In this manner, the detection device 1 can be downsized in comparison with the case where the biological information detection part 15 and the acceleration detection part 12 are housed in separate housing parts. That is, the detection device 1 can suppress the heating of the acceleration detection part 12 due to incidence of light while reducing the size. Further, in the detection device 1 illustrated in FIGS. 11 and 12, the control part 14 is provided between the light emission part 151 and the light reception part 152 in the direction in which the light emission part 151 and the light reception part 152 are arranged. In this manner, the detection device 1 can reduce the amount of the stray light incident on the light reception part 152 while achieving size reception.

### Sixth Modification of First Embodiment

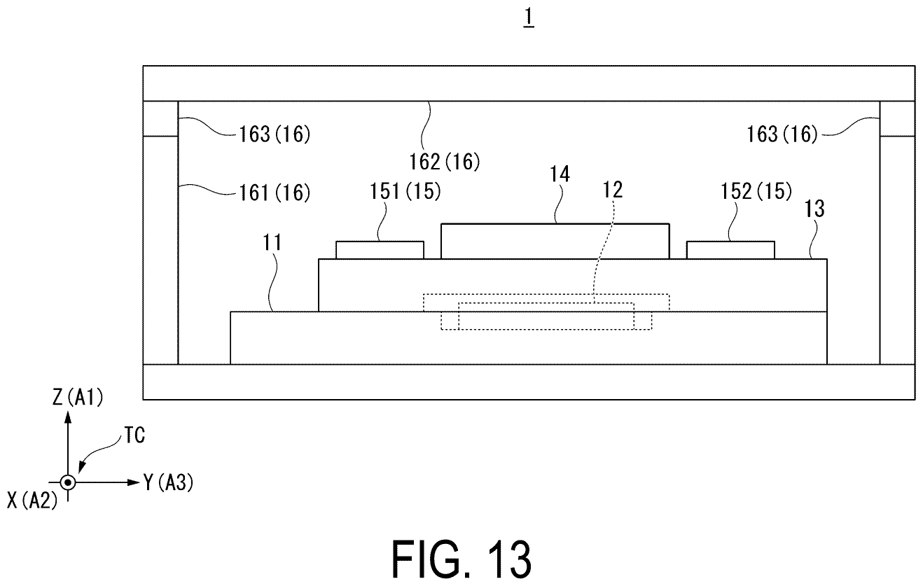

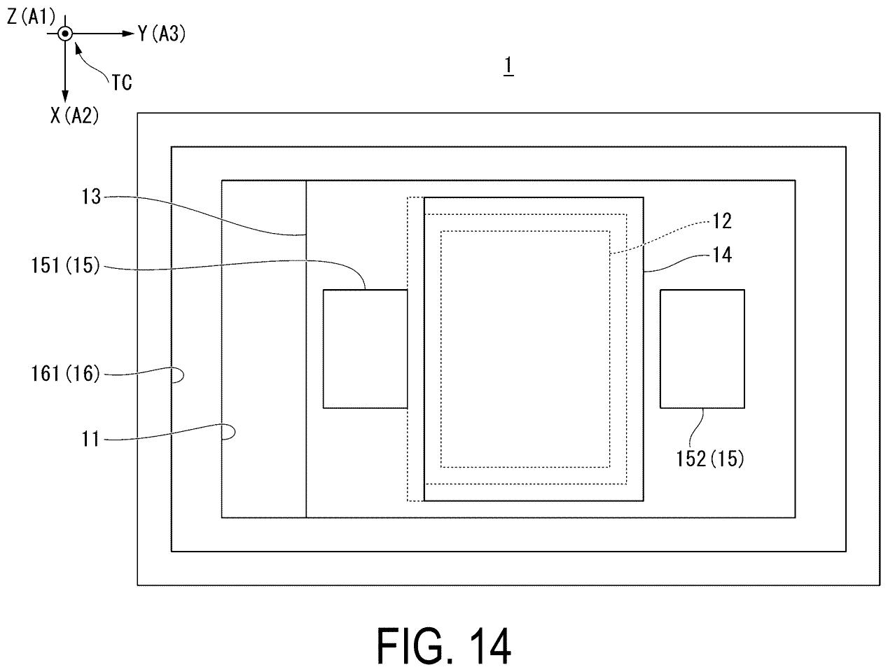

In a sixth modification of the first embodiment, the light emission part 151 and the light reception part 152 are both provided on the lid part 13, the light emission part 151 is disposed side by side with the light reception part 152 and the control part 14 in the direction A3, and the control part 14 is located between the light emission part 151 and the light reception part 152 in the direction A3. FIG. 13 is a front view illustrating a sixth modification of the configuration of the detection device 1. FIG. 14 is a top view of the detection device 1 illustrated in FIG. 13. Note that in FIG. 13, to clearly illustrate the inner configuration of the detection device 1, the member that covers the front surface of the inside of the detection device 1 is omitted. In addition, in FIG. 14, to clearly illustrate the inner configuration of the detection device 1, the member that covers the top surface of the inside of the detection device 1 is omitted.

In the example illustrated in FIGS. 11 and 12, the light emission part 151 and the light reception part 152 are both provided on the lid part 13 as in the example illustrated in FIGS. 3 and 4. However, unlike in the first modification of the first embodiment, in FIGS. 13 and 14, the light emission part 151 is not disposed side by side with the light reception part 152 in the direction A2, but is disposed side by side with the control part 14 and the light reception part 152 in the order of the light emission part 151, the control part 14, and the light reception part 152 in the direction A3. Specifically, in this example, the control part 14 is located between the light emission part 151 and the light reception part 152 in the direction A3.

Here, also in the example illustrated in FIGS. 13 and 14, the height of the top surface of the light emission part 151 is greater than the height of the bottom surface of the lid part 13. Therefore, also in this case, in the detection device 1, the amount of light emitted from the light emission part 151 to the acceleration detection part 12 can be reduced.

Specifically, as long as the height of the top surface of the light emission part 151 is greater than the height of the bottom surface of the lid part 13, the detection device 1 can suppress the heating of the acceleration detection part 12 due to incidence of light even when the light emission part 151 and the light reception part 152 are disposed side by side with the control part 14 therebetween on the lid part 13.

In addition, also in the example illustrated in FIGS. 13 and 14, the light emission part 151 and the light reception part 152 are provided with the control part 14 therebetween on the lid part 13. In this manner, in this example, the light that is emitted from the light emission part 151 and travels toward the light reception part 152 is reflected by the control part 14, and is less likely to be received by the light reception part 152 as stray light. That is, with the control part 14 provided between the light emission part 151 and the light reception part 152 in the direction in which the light emission part 151 and the light reception part 152 are arranged in the housing part 16, the detection device 1 can reduce the amount of the stray light incident on the light reception part 152 while achieving size reception. In addition, the detection device 1 can reduce the amount of the stray light incident on the light reception part 152 in this manner, and therefore does not need to include a light blocking member for blocking light to prevent stray light incident on the light reception part 152. As a result, the detection device 1 can be more reliably downsized.

As described above, also in the detection device 1 illustrated in FIGS. 13 and 14, the height of the top surface of the light emission part 151 is equal to or greater than the height of the bottom surface of the lid part 13. In this manner, the detection device 1 can reduce the amount of light emitted from the light emission part 151 to the acceleration detection part 12. As a result, the detection device 1 can suppress the heating of the acceleration detection part 12 due to incidence of light. In addition, also in the detection device 1 illustrated in FIGS. 13 and 14, the acceleration detection part 12 and the biological information detection part 15 are housed in the housing part 16. In this manner, the detection device 1 can be downsized in comparison with the case where the biological information detection part 15 and the acceleration detection part 12 are housed in separate housing parts. That is, the detection device 1 can suppress the heating of the acceleration detection part 12 due to incidence of light while reducing the size. Further, also in the detection device 1 illustrated in FIGS. 13 and 14, the control part 14 is provided between the light emission part 151 and the light reception part 152 in the direction in which the light emission part 151 and the light reception part 152 are arranged. In this manner, the detection device 1 can reduce the amount of the stray light incident on the light reception part 152 while achieving size reception.

### Seventh Modification of First Embodiment

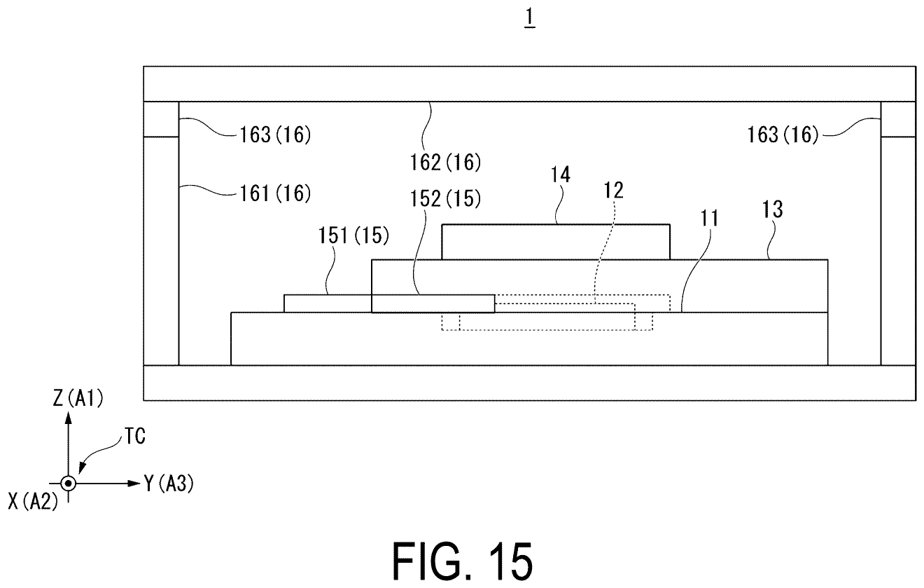

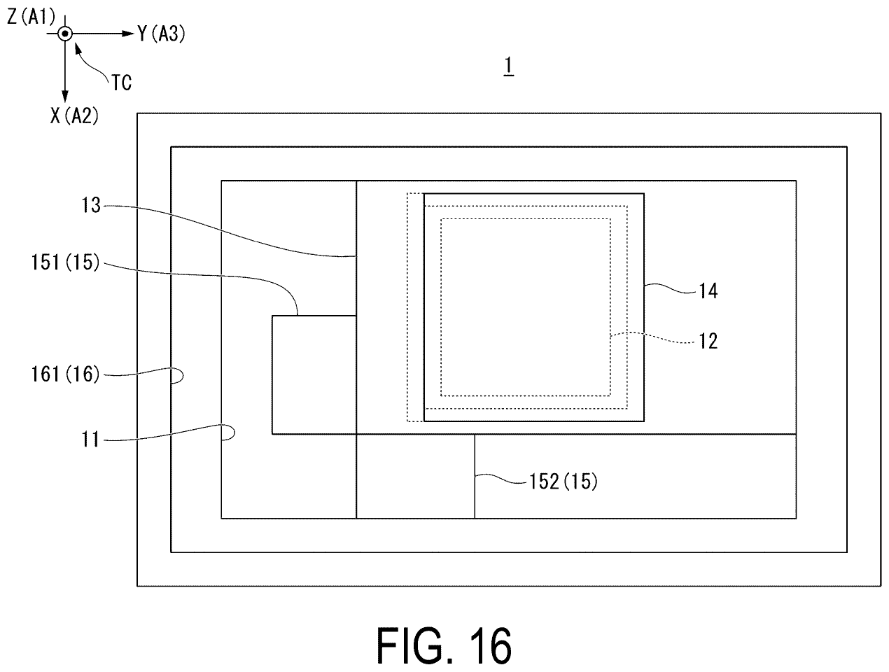

In a seventh modification of the first embodiment, the light emission part 151 and the light reception part 152 are both provided on the base part 11, and the light emission part 151 is disposed side by side with the control part 14 in the direction A3, and the light reception part 152 is disposed side by side with the control part 14 in the direction A2. FIG. 15 is a front view illustrating the seventh modification of the configuration of the detection device 1. FIG. 16 is a top view of the detection device 1 illustrated in FIG. 15. Note that in FIG. 15, to clearly illustrate the inner configuration of the detection device 1, the member that covers the front surface of the inside of the detection device 1 is omitted. In addition, in FIG. 16, to clearly illustrate the inner configuration of the detection device 1, the member that covers the top surface of the inside of the detection device 1 is omitted.

In the example illustrated in FIGS. 15 and 16, the light emission part 151 and the light reception part 152 are both provided on the base part 11 as in the example illustrated in FIGS. 1 and 2. However, unlike in the first embodiment, in FIGS. 15 and 16, the light emission part 151 is not disposed side by side with the light reception part 152 in the direction A2, but is disposed side by side with the control part 14 in the direction A3. In addition, in FIGS. 15 and 16, the light reception part 152 is disposed side by side with the control part 14 in the direction A2.

Here, also in the example illustrated in FIGS. 15 and 16, the height of the top surface of the light emission part 151 is greater than the height of the bottom surface of the lid part 13. Therefore, also in this case, in the detection device 1, the amount of light emitted from the light emission part 151 to the acceleration detection part 12 can be reduced.

Specifically, as long as the height of the top surface of the light emission part 151 is greater than the height of the bottom surface of the lid part 13, the detection device 1 can suppress the heating of the acceleration detection part 12 due to incidence of light even when the light emission part 151 is disposed side by side with the control part 14 in the direction A3, and the light reception part 152 is disposed side by side with the control part 14 in the direction A2.

In addition, in the example illustrated in FIGS. 15 and 16, the light emission part 151 does not overlap both the lid part 13 and the light reception part 152 as viewed toward the direction A2. On the other hand, in this example, the light reception part 152 overlaps the lid part 13 as viewed toward the direction A2. In addition, in this example, the light emission part 151 overlaps the lid part 13 but does not overlap the light reception part 152 as viewed toward the direction A1. In this manner, at least a part of the light that is emitted from the light emission part 151 and travels toward the light reception part 152 is reflected by the lid part 13, and is less likely to be received by the light reception part 152 as stray light. Specifically, since the biological information detection part 15 and the acceleration detection part 12 are provided in the housing part 16, the light emission part 151 is disposed side by side with the control part 14 in the direction A3, and the light reception part 152 is disposed side by side with the control part 14 in the direction A2, the detection device 1 can reduce the amount of the stray light incident on the light reception part 152 while achieving size reception. Note that this effect increases as the distance between the light emission part 151 and the light reception part 152 increases. Therefore, in this example, the larger the distance between the light emission part 151 and the light reception part 152, the more desirable.

As described above, also in the detection device 1 illustrated in FIGS. 15 and 16, the height of the top surface of the light emission part 151 is equal to or greater than the height of the bottom surface of the lid part 13. In this manner, the detection device 1 can reduce the amount of light emitted from the light emission part 151 to the acceleration detection part 12. As a result, the detection device 1 can suppress the heating of the acceleration detection part 12 due to incidence of light. In addition, also in the detection device 1 illustrated in FIGS. 15 and 16, the acceleration detection part 12 and the biological information detection part 15 are housed in the housing part 16. In this manner, the detection device 1 can be downsized in comparison with the case where the biological information detection part 15 and the acceleration detection part 12 are housed in separate housing parts. That is, the detection device 1 can suppress the heating of the acceleration detection part 12 due to incidence of light while reducing the size. Further, in the detection device 1 illustrated in FIGS. 15 and 16, at least a part of the light that is emitted from the light emission part 151 and travels toward the light reception part 152 is reflected by the lid part 13, and is less likely to be received by the light reception part 152 as stray light. In this manner, the detection device 1 can reduce the amount of the stray light incident on the light reception part 152 while achieving size reception.

### Eighth Modification of First Embodiment

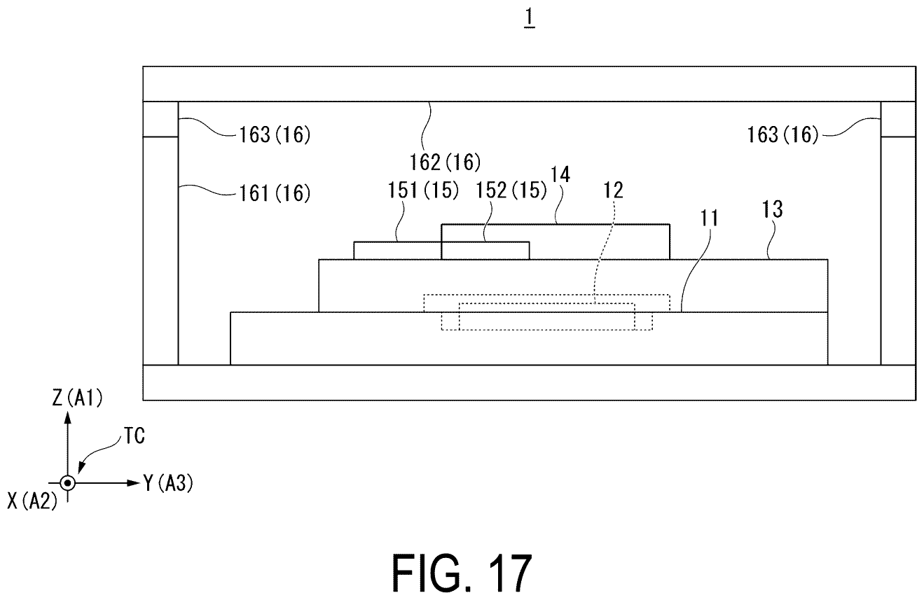

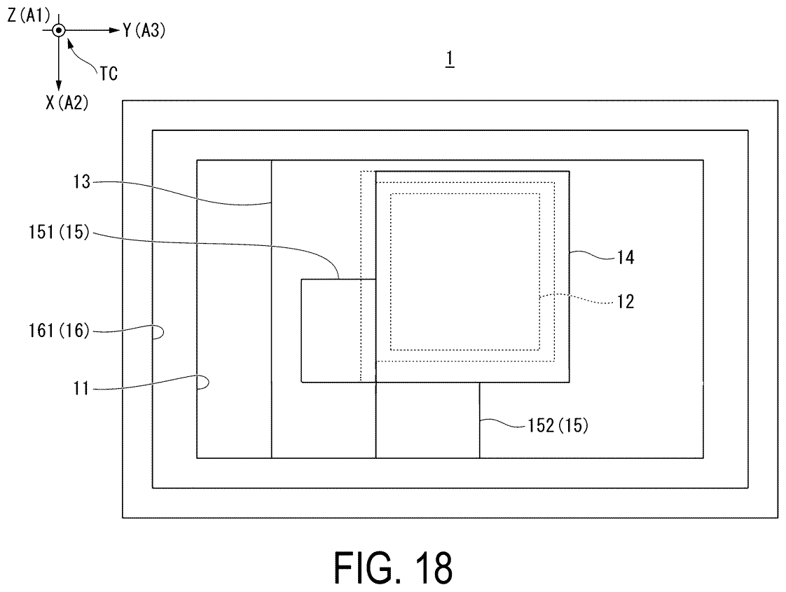

In an eighth modification of the first embodiment, the light emission part 151 and the light reception part 152 are both provided on the lid part 13, the light emission part 151 is disposed side by side with the control part 14 in the direction A3, and the light reception part 152 is disposed side by side with the control part 14 in the direction A2. FIG. 17 is a front view illustrating the eighth modification of the configuration of the detection device 1. FIG. 18 is a top view of the detection device 1 illustrated in FIG. 17. Note that in FIG. 17, to clearly illustrate the inner configuration of the detection device 1, the member that covers the front surface of the inside of the detection device 1 is omitted. In addition, in FIG. 18, to clearly illustrate the inner configuration of the detection device 1, the member that covers the top surface of the inside of the detection device 1 is omitted.

In the example illustrated in FIGS. 17 and 18, the light emission part 151 and the light reception part 152 are both provided on the lid part 13 as in the example illustrated in FIGS. 3 and 4. However, unlike in the first modification of the first embodiment, in FIGS. 17 and 18, the light emission part 151 is not disposed side by side with the light reception part 152 in the direction A2, but is disposed side by side with the control part 14 in the direction A3. In addition, in FIGS. 17 and 18, the light reception part 152 is disposed side by side with the control part 14 in the direction A2.

Here, also in the example illustrated in FIGS. 17 and 18, the height of the top surface of the light emission part 151 is greater than the height of the bottom surface of the lid part 13. Therefore, also in this case, in the detection device 1, the amount of light emitted from the light emission part 151 to the acceleration detection part 12 can be reduced.

Specifically, as long as the height of the top surface of the light emission part 151 is greater than the height of the bottom surface of the lid part 13, the detection device 1 can suppress the heating of the acceleration detection part 12 due to incidence of light even when the light emission part 151 is disposed side by side with the control part 14 in the direction A3, and the light reception part 152 is disposed side by side with the control part 14 in the direction A2.

In addition, in the example illustrated in FIGS. 17 and 18, the light emission part 151 does not overlap both the control part 14 and the light reception part 152 as viewed toward the direction A2. On the other hand, in this example, the light reception part 152 overlaps the control part 14 as viewed toward the direction A2. In addition, in this example, the light emission part 151 overlaps the control part 14 but does not overlap the light reception part 152 as viewed toward the direction A1. In this manner, at least a part of the light that is emitted from the light emission part 151 and travels toward the light reception part 152 is reflected by the control part 14, and is less likely to be received by the light reception part 152 as stray light. Specifically, since the biological information detection part 15 and the acceleration detection part 12 are provided in the housing part 16, the light emission part 151 is disposed side by side with the control part 14 in the direction A3, and the light reception part 152 is disposed side by side with the control part 14 in the direction A2, the detection device 1 can reduce the amount of the stray light incident on the light reception part 152 while achieving size reception.

As described above, also in the detection device 1 illustrated in FIGS. 17 and 18, the height of the top surface of the light emission part 151 is equal to or greater than the height of the bottom surface of the lid part 13. In this manner, the detection device 1 can reduce the amount of light emitted from the light emission part 151 to the acceleration detection part 12. As a result, the detection device 1 can suppress the heating of the acceleration detection part 12 due to incidence of light. In addition, also in the detection device 1 illustrated in FIGS. 17 and 18, the acceleration detection part 12 and the biological information detection part 15 are housed in the housing part 16. In this manner, the detection device 1 can be downsized in comparison with the case where the biological information detection part 15 and the acceleration detection part 12 are housed in separate housing parts. That is, the detection device 1 can suppress the heating of the acceleration detection part 12 due to incidence of light while reducing the size. Further, in the detection device 1 illustrated in FIGS. 17 and 18, at least a part of the light that is emitted from the light emission part 151 and travels toward the light reception part 152 is reflected by the control part 14, and is less likely to be received by the light reception part 152 as stray light. In this manner, the detection device 1 can reduce the amount of the stray light incident on the light reception part 152 while achieving size reception.

Note that in each of the first embodiment and the first to eighth modifications of the first embodiment described above, the positional relationship between the light emission part 151 and the light reception part 152 may be reversed. In addition, in each of the first embodiment and the first to eighth modifications of the first embodiment described above, the shapes of the light emission part 151 and the light reception part 152 may be any shapes, and may be the same shape or different shapes. In addition, the first embodiment and the first to eighth modifications of the first embodiment described above may be combined with each other in any way.

### Second Embodiment

A second embodiment is described below with reference to the accompanying drawings.

### Overview of Detection Device According To Second Embodiment

First, an overview of a detection device according to a second embodiment is described.

A detection device according to the second embodiment is attached to a predetermined portion of a subject person. In the detection device, a predetermined first direction is set as an upward direction. In addition, the detection device includes a base part, an acceleration detection part, a lid part, a control part, a light emission part, a light reception part, and a housing part. The base part is a surface orthogonal to the first direction. The acceleration detection part is provided on the base part. The lid part covers the acceleration detection part together with the base part on the base part. The control part is provided on the lid part, and configured to detect the body movement based on the output of the acceleration detection part when the detection device is attached to the portion subject person. The light emission part emits light. The light reception part receives light. The housing part houses the base part, the acceleration detection part, the lid part, the control part, the light emission part and the light reception part. Further, the height of the top surface of the light emission part in the first direction is different from the height of the top surface of the light reception part in the first direction. In addition, the light emission part and the light reception part are provided on different members. In this manner, the detection device 2 can suppress a situation where at least a part of the light that is emitted from the light emission part and travels toward the light reception part directly impinges on the reception part so as to be received by the light reception part.

A configuration of the detection device according to the second embodiment is elaborated below. Note that in the second embodiment, the same components as those of the first embodiment are denoted with the same reference numerals, and the description thereof is omitted.

### Configuration of Detection Device According To Second Embodiment

A configuration of the detection device according to the second embodiment is described below with a detection device 2 as an example. In the second embodiment, viewing the detection device 2 toward a certain direction is referred to as viewing from that direction for convenience of description.

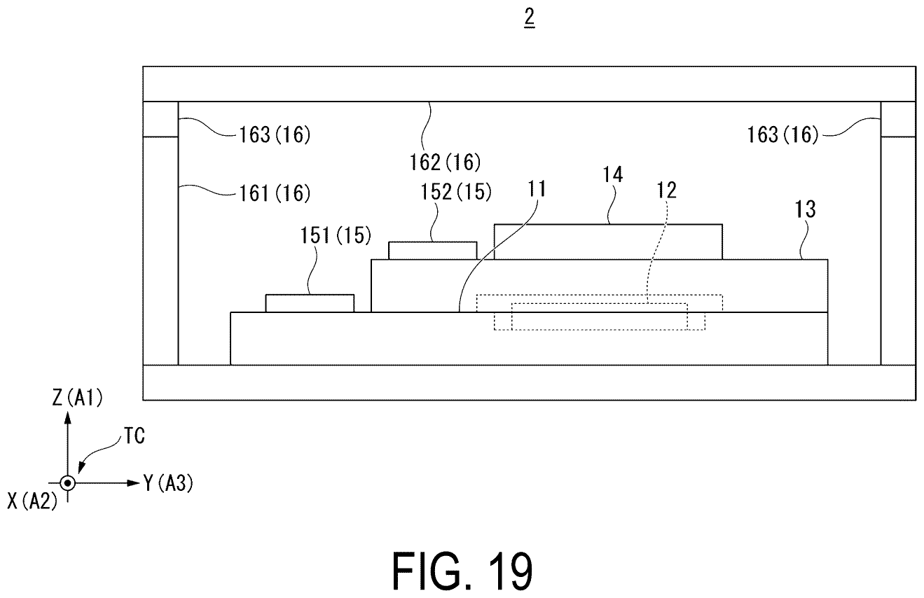

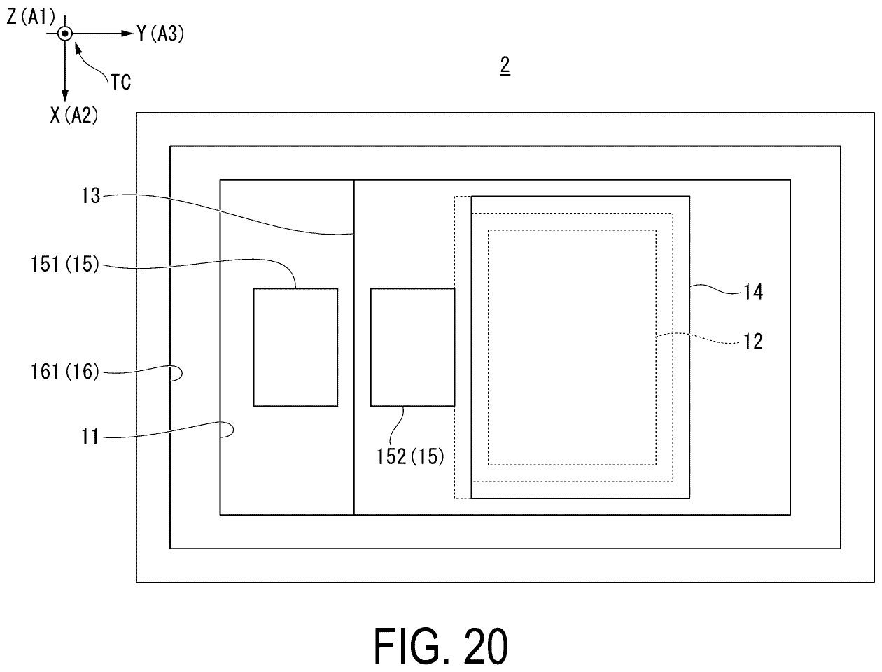

The detection device 2 has the same configuration as that of the detection device 1 except that the light emission part 151 and the light reception part 152 are provided on different members. FIG. 19 is a front view illustrating an example of the configuration of the detection device 2. FIG. 20 is a top view of the detection device 2 illustrated in FIG. 19. Note that in FIG. 19, to clearly illustrate the inner configuration of the detection device 2, the member that covers the front surface of the inside of the detection device 2 is omitted. In addition, in FIG. 20, to clearly illustrate the inner configuration of the detection device 2, the member that covers the top surface of the inside of the detection device 2 is omitted.

In the example illustrated in FIGS. 19 and 20, the light emission part 151 is provided on the base part 11. On the other hand, in this example, the light reception part 152 is provided on the lid part 13. As such, in this example, the height of the top surface of the light reception part 152 is greater than the height of the top surface of the light emission part 151. As a result, in the detection device 2, at least a part of the light that is emitted from the light emission part 151 and travels toward the light reception part 152 are reflected at the side surface of the lid part 13 and the side surface of the light reception part 152, and does not reach the light-receiving element provided in the light reception part 152. In this manner, the detection device 2 can suppress a situation where at least a part of the light that is emitted from the light emission part 151 and travels toward the light reception part 152 directly impinges on the light reception part 152 so as to be received by the light reception part 152.

In addition, in the example illustrated in FIGS. 19 and 20, the positional relationship between the light emission part 151 and the light reception part 152 may be reversed. In this case, the height of the top surface of the light emission part 151 is greater than the height of the top surface of the light reception part 152. As a result, in the detection device 2, most of the light emitted from the light emission part 151 travels upward without travelling toward the light reception part 152. Even in this manner, the detection device 2 can suppress a situation where at least a part of the light that is emitted from the light emission part 151 and travels toward the light reception part 152 directly impinges on the light reception part 152 so as to be received by the light reception part 152.

Here, in the example illustrated in FIGS. 19 and 20, the light emission part 151, the light reception part 152 and the control part 14 are disposed side by side in the order of the light emission part 151, the light reception part 152 and the control part 14 in the direction A3. However, in the detection device 2, the effect of suppressing a situation where at least a part of the light that is emitted from the light emission part 151 and travels toward the light reception part 152 is received by the light reception part 152 is achieved by the height of the top surface of the light reception part 152 that is different from the height of the top surface of the light emission part 151 regardless of the order of the light emission part 151, the light reception part 152 and the control part 14 in the direction A3.

Note that the light emission part 151 may be provided on the base part 11 with a spacer or the like therebetween. In addition, the light reception part 152 may be provided on the lid part 13 with a spacer or the like therebetween.

Also in the example illustrated in FIGS. 19 and 20, the height of the top surface of the light emission part 151 is greater than the height of the bottom surface of the lid part 13. Therefore, also in this case, in the detection device 2, the amount of light emitted from the light emission part 151 to the acceleration detection part 12 can be reduced. As a result, the detection device 2 can suppress the heating of the acceleration detection part 12 due to incidence of light.

In addition, also in the example illustrated in FIGS. 19 and 20, the housing part 16 houses the biological information detection part 15 together with the acceleration detection part 12. In this manner, the detection device 2 can be downsized in comparison with the case where the biological information detection part 15 and the acceleration detection part 12 are housed in separate housing parts.

In addition, in the example illustrated in FIGS. 19 and 20, the height of the top surface of the light emission part 151 and the height of the top surface of the light reception part 152 are both lower than the height of the top surface of the control part 14. In this case, the thickness of the detection device 2 in the direction A1 can be reduced, and thus the detection device 2 can be downsized as a whole. To obtain this effect, the height of the top surface of the light emission part 151 and the height of the top surface of the light reception part 152 may both be the same as the height of the top surface of the control part 14.

As described above, in the detection device 2 illustrated in FIGS. 19 and 20, the height of the top surface of the light emission part 151 is different from the height of the top surface of the light reception part 152. In this manner, the detection device 2 can suppress a situation where at least a part of the light that is emitted from the light emission part 151 and travels toward the light reception part 152 is received by the light reception part 152. In addition, also in the detection device 2 illustrated in FIGS. 19 and 20, the acceleration detection part 12 and the biological information detection part 15 are housed in the housing part 16. In this manner, the detection device 2 can be downsized in comparison with the case where the biological information detection part 15 and the acceleration detection part 12 are housed in separate housing parts. That is, the detection device 2 can reduce the amount of the stray light incident on the light reception part 152 while achieving size reception.

### First Modification of Second Embodiment

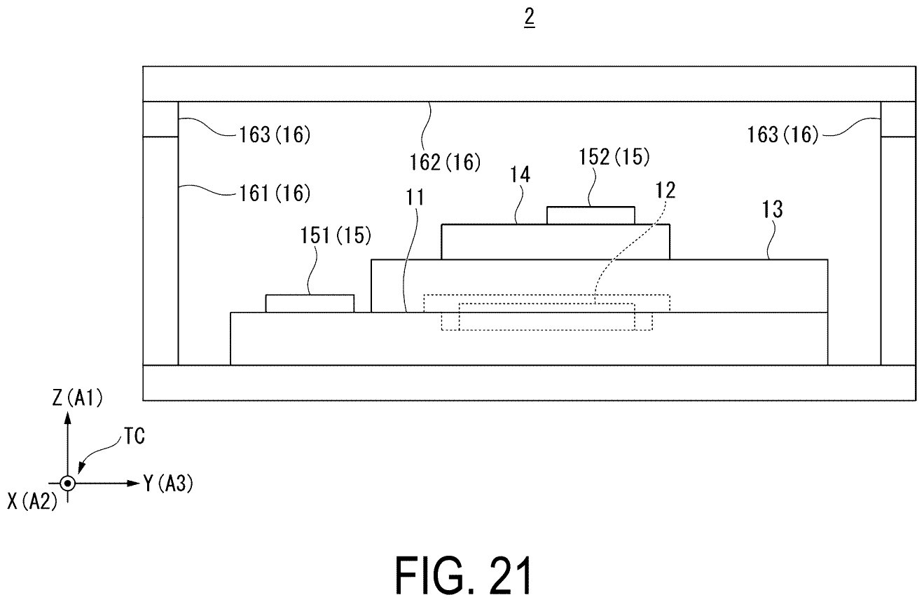

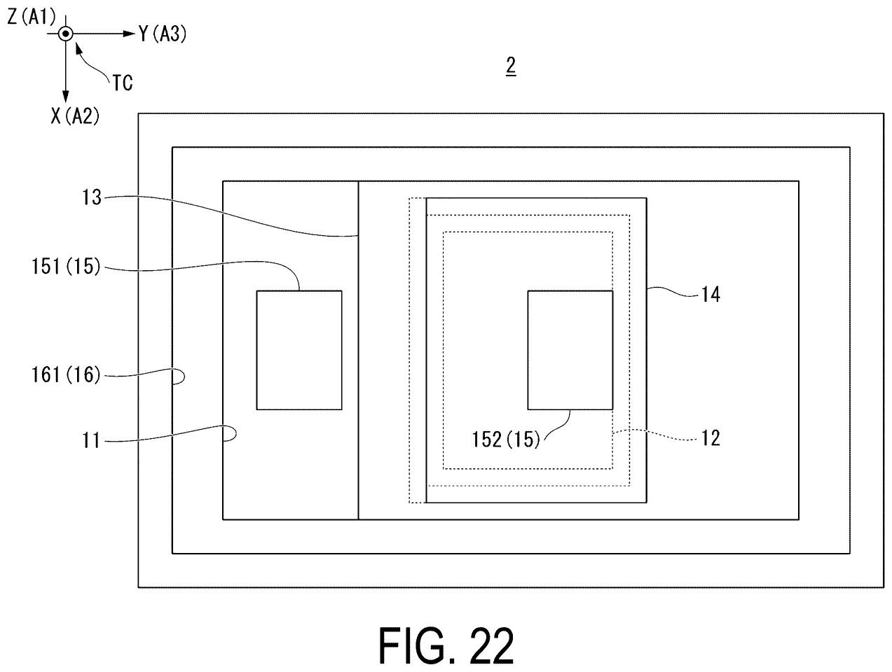

In a first modification of the second embodiment, the light emission part 151 is provided on the base part 11, while the light reception part 152 is provided on the control part 14. FIG. 21 is a front view illustrating the first modification of the configuration of the detection device 2. FIG. 22 is a top view of the detection device 2 illustrated in FIG. 21. Note that in FIG. 21, to clearly illustrate the inner configuration of the detection device 2, the member that covers the front surface of the inside of the detection device 2 is omitted. In addition, in FIG. 22, to clearly illustrate the inner configuration of the detection device 2, the member that covers the top surface of the inside of the detection device 2 is omitted.

In the example illustrated in FIGS. 21 and 22, the light emission part 151 is provided on the base part 11 as in the second embodiment. On the other hand, in this example, the light reception part 152 is provided on the control part 14. As such, also in this example, the height of the top surface of the light reception part 152 is greater than the height of the top surface of the light emission part 151. As a result, in the detection device 2, at least a part of the light that is emitted from the light emission part 151 and travels toward the light reception part 152 is reflected at the side surface of the lid part 13, the side surface of the control part 14, and the side surface of the light reception part 152, and does not reach the light-receiving element provided in the light reception part 152. In this manner, the detection device 2 can suppress a situation where at least a part of the light that is emitted from the light emission part 151 and travels toward the light reception part 152 directly impinges on the light reception part 152 so as to be received by the light reception part 152.

In addition, in the example illustrated in FIGS. 21 and 22, the positional relationship between the light emission part 151 and the light reception part 152 may be reversed. In this case, the height of the top surface of the light emission part 151 is greater than the height of the top surface of the light reception part 152. As a result, in the detection device 2, most of the light emitted from the light emission part 151 travels upward without travelling toward the light reception part 152. Even in this manner, the detection device 2 can suppress a situation where at least a part of the light that is emitted from the light emission part 151 and travels toward the light reception part 152 is received by the light reception part 152.

Here, also in the example illustrated in FIGS. 21 and 22, the light emission part 151, the light reception part 152 and the control part 14 are disposed side by side in the order of the light emission part 151, the light reception part 152 and the control part 14 in the direction A3. However, in the detection device 2, the effect of suppressing a situation where at least a part of the light that is emitted from the light emission part 151 and travels toward the light reception part 152 is received by the light reception part 152 is achieved by the height of the top surface of the light reception part 152 that is different from the height of the top surface of the light emission part 151 regardless of the order of the light emission part 151, the light reception part 152 and the control part 14 in the direction A3.

Note that the light emission part 151 may be provided on the base part 11 with a spacer or the like therebetween. In addition, the light reception part 152 may be provided on the control part 14 with a spacer or the like therebetween.

Also in the example illustrated in FIGS. 21 and 22, the height of the top surface of the light emission part 151 is greater than the height of the bottom surface of the lid part 13. Therefore, also in this case, in the detection device 2, the amount of light emitted from the light emission part 151 to the acceleration detection part 12 can be reduced. As a result, the detection device 2 can suppress the heating of the acceleration detection part 12 due to incidence of light.

In addition, also in the example illustrated in FIGS. 21 and 22, the housing part 16 houses the biological information detection part 15 together with the acceleration detection part 12. In this manner, the detection device 2 can be downsized in comparison with the case where the biological information detection part 15 and the acceleration detection part 12 are housed in separate housing parts.

As described above, also in the detection device 2 illustrated in FIGS. 21 and 22, the height of the top surface of the light emission part 151 is different from the height of the top surface of the light reception part 152. In this manner, the detection device 2 can suppress a situation where at least a part of the light that is emitted from the light emission part 151 and travels toward the light reception part 152 is received by the light reception part 152. In addition, also in the detection device 2 illustrated in FIGS. 21 and 22, the acceleration detection part 12 and the biological information detection part 15 are housed in the housing part 16. In this manner, the detection device 2 can be downsized in comparison with the case where the biological information detection part 15 and the acceleration detection part 12 are housed in separate housing parts. That is, the detection device 2 can reduce the amount of the stray light incident on the light reception part 152 while achieving size reception.

### Second Modification of Second Embodiment

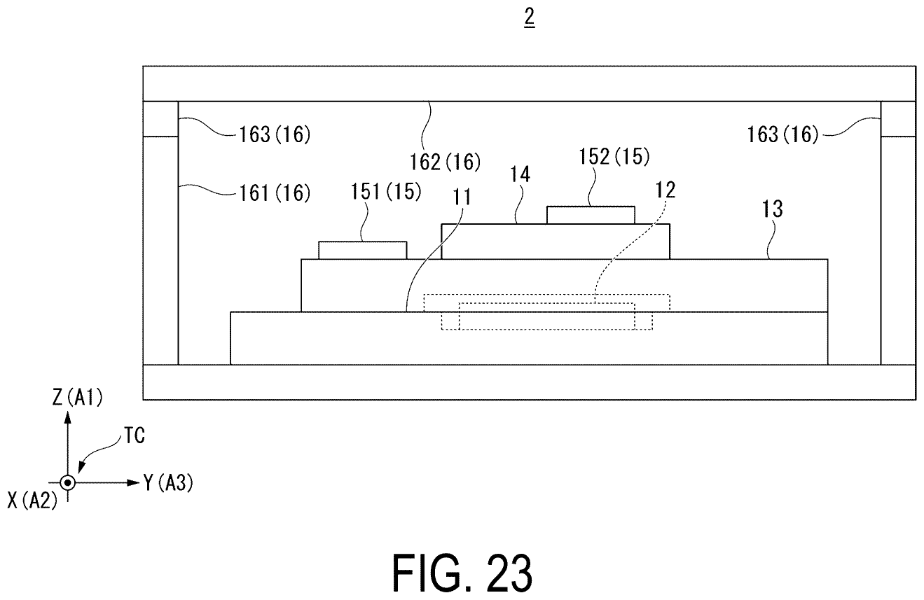

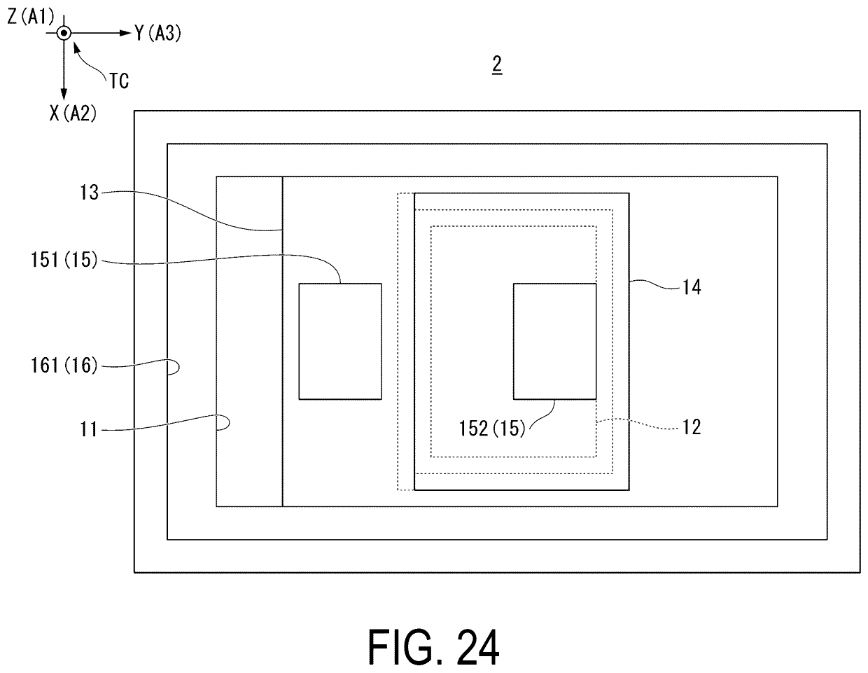

In a second modification of the second embodiment, the light emission part 151 is provided on the lid part 13, while the light reception part 152 is provided on the control part 14. FIG. 23 is a front view illustrating a second modification of the configuration of the detection device 2. FIG. 24 is a top view of the detection device 2 illustrated in FIG. 23. Note that in FIG. 23, to clearly illustrate the inner configuration of the detection device 2, the member that covers the front surface of the inside of the detection device 2 is omitted. In addition, in FIG. 24, to clearly illustrate the inner configuration of the detection device 2, the member that covers the top surface of the inside of the detection device 2 is omitted.

In the example illustrated in FIGS. 23 and 24, the light emission part 151 is provided on the lid part 13. On the other hand, in this example, the light reception part 152 is provided on the control part 14 as in the second embodiment. As such, also in this example, the height of the top surface of the light reception part 152 is greater than the height of the top surface of the light emission part 151. As a result, in the detection device 2, at least a part of the light that is emitted from the light emission part 151 and travels toward the light reception part 152 is reflected at the side surface of the lid part 13, the side surface of the control part 14, and the side surface of the light reception part 152, and does not reach the light-receiving element provided in the light reception part 152. In this manner, the detection device 2 can suppress a situation where at least a part of the light that is emitted from the light emission part 151 and travels toward the light reception part 152 directly impinges on the light reception part 152 so as to be received by the light reception part 152.

In addition, also in the example illustrated in FIGS. 23 and 24, the positional relationship between the light emission part 151 and the light reception part 152 may be reversed. In this case, the height of the top surface of the light emission part 151 is greater than the height of the top surface of the light reception part 152. As a result, in the detection device 2, most of the light emitted from the light emission part 151 travels upward without travelling toward the light reception part 152. Even in this manner, the detection device 2 can suppress a situation where at least a part of the light that is emitted from the light emission part 151 and travels toward the light reception part 152 directly impinges on the light reception part 152 so as to be received by the light reception part 152.

Here, also in the example illustrated in FIGS. 23 and 24, the light emission part 151 and the light reception part 152 are disposed side by side in the order of the light emission part 151 and the light reception part 152 in the direction A3. However, in the detection device 2, the effect of suppressing a situation where at least a part of the light that is emitted from the light emission part 151 and travels toward the light reception part 152 directly impinges on the light reception part 152 so as to be received by the light reception part 152 is achieved by the height of the top surface of the light reception part 152 that is different from the height of the top surface of the light emission part 151 regardless of the order of the light emission part 151 and the light reception part 152 in the direction A3.

Note that the light emission part 151 may be provided on the base part 11 with a spacer or the like therebetween. In addition, the light reception part 152 may be provided on the control part 14 with a spacer or the like therebetween.

In addition, also in the example illustrated in FIGS. 23 and 24, the housing part 16 houses the biological information detection part 15 together with the acceleration detection part 12. Therefore, also in this case, in the detection device 2, the amount of light emitted from the light emission part 151 to the acceleration detection part 12 can be reduced. As a result, the detection device 2 can suppress the heating of the acceleration detection part 12 due to incidence of light.

As described above, also in the detection device 2 illustrated in FIGS. 23 and 24, the height of the top surface of the light emission part 151 is different from the height of the top surface of the light reception part 152. In this manner, the detection device 2 can be downsized in comparison with the case where the biological information detection part 15 and the acceleration detection part 12 are housed in separate housing parts.

As described above, also in the detection device 2 illustrated in FIGS. 23 and 24, the height of the top surface of the light emission part 151 is different from the height of the top surface of the light reception part 152. In this manner, the detection device 2 can suppress a situation where at least a part of the light that is emitted from the light emission part 151 and travels toward the light reception part 152 is received by the light reception part 152. In addition, also in the detection device 2 illustrated in FIGS. 23 and 24, the acceleration detection part 12 and the biological information detection part 15 are housed in the housing part 16. In this manner, the detection device 2 can be downsized in comparison with the case where the biological information detection part 15 and the acceleration detection part 12 are housed in separate housing parts. That is, the detection device 2 can reduce the amount of the stray light incident on the light reception part 152 while achieving size reception.

### Third Modification of Second Embodiment

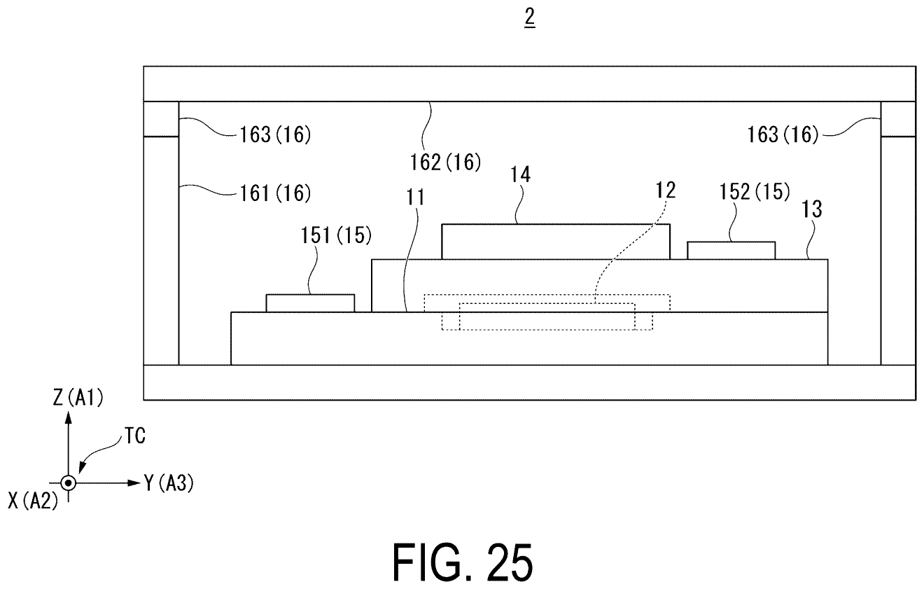

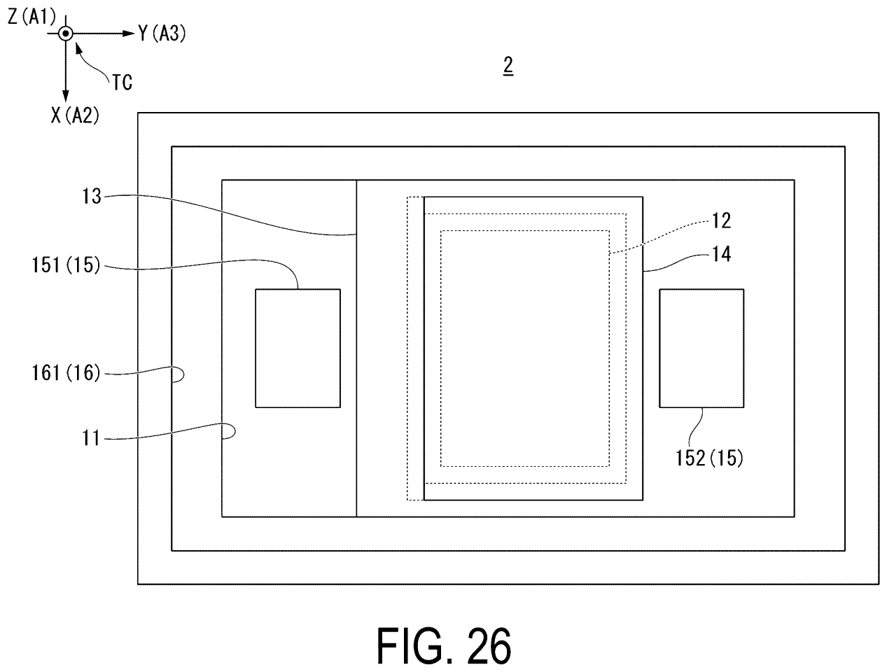

In a third modification of the second embodiment, as in the second embodiment, the light emission part 151 is provided on the base part 11, and the light reception part 152 is provided on the lid part 13. On the other hand, in the third modification of the second embodiment, unlike in the second embodiment, the control part 14 is located between the light emission part 151 and the light reception part 152 in the direction A3. FIG. 25 is a front view illustrating the third modification of the configuration of the detection device 2. FIG. 26 is a top view of the detection device 2 illustrated in FIG. 25. Note that in FIG. 25, to clearly illustrate the inner configuration of the detection device 2, the member that covers the front surface of the inside of the detection device 2 is omitted. In addition, in FIG. 26, to clearly illustrate the inner configuration of the detection device 2, the member that covers the top surface of the inside of the detection device 2 is omitted.

In the example illustrated in FIGS. 25 and 26, the light emission part 151 is provided on the base part 11. On the other hand, in this example, the light reception part 152 is provided on the lid part 13. As such, also in this example, the height of the top surface of the light reception part 152 is greater than the height of the top surface of the light emission part 151. In addition, in this example, the light emission part 151, the light reception part 152 and the control part 14 are disposed side by side in the order of the light emission part 151, the control part 14, and the light reception part 152 in the direction A3. That is, the control part 14 is located between the light emission part 151 and the light reception part 152 in the direction A3. In this case, the light that is emitted from the light emission part 151 and travels toward the light reception part 152 does not reach the light reception part 152 unless it is subjected to reflection, refraction, diffraction or the like. Thus, in this example, the detection device 2 can more reliably suppress a situation where at least a part of the light that is emitted from the light emission part 151 and travels toward the light reception part 152 directly impinges on the light reception part 152 so as to be received by the light reception part 152.

In addition, also in the example illustrated in FIGS. 25 and 26, the positional relationship between the light emission part 151 and the light reception part 152 may be reversed. In this case, the height of the top surface of the light emission part 151 is greater than the height of the top surface of the light reception part 152. As a result, in the detection device 2, the light emitted from the light emission part 151 travels upward without travelling toward the light reception part 152 unless it is subjected to reflection, refraction, diffraction or the like. Even in this manner, the detection device 2 can suppress a situation where at least a part of the light that is emitted from the light emission part 151 and travels toward the light reception part 152 directly impinges on the light reception part 152 so as to be received by the light reception part 152.

Note that the light emission part 151 may be provided on the base part 11 with a spacer or the like therebetween. In addition, the light reception part 152 may be provided on the control part 14 with a spacer or the like therebetween.

In addition, in the example illustrated in FIGS. 25 and 26, the height of the top surface of the light emission part 151 and the height of the top surface of the light reception part 152 are both lower than the height of the top surface of the control part 14. In this case, the thickness of the detection device 2 in the direction A1 can be reduced, and thus the detection device 2 can be downsized as a whole. To obtain this effect, the height of the top surface of the light emission part 151 and the height of the top surface of the light reception part 152 may both be the same as the height of the top surface of the control part 14.

In addition, also in the example illustrated in FIGS. 25 and 26, the height of the top surface of the light emission part 151 is greater than the height of the bottom surface of the lid part 13. Therefore, also in this case, in the detection device 2, the amount of light emitted from the light emission part 151 to the acceleration detection part 12 can be reduced. As a result, the detection device 2 can suppress the heating of the acceleration detection part 12 due to incidence of light.

In addition, also in the example illustrated in FIGS. 25 and 26, the housing part 16 houses the biological information detection part 15 together with the acceleration detection part 12. In this manner, the detection device 2 can be downsized in comparison with the case where the biological information detection part 15 and the acceleration detection part 12 are housed in separate housing parts.

As described above, also in the detection device 2 illustrated in FIGS. 25 and 26, the height of the top surface of the light emission part 151 is different from the height of the top surface of the light reception part 152. In addition, in the detection device 2 illustrated in FIGS. 25 and 26, the control part 14 is located between the light emission part 151 and the light reception part 152 in the direction A3. In this manner, the detection device 2 can more reliably suppress a situation where at least a part of the light that is emitted from the light emission part 151 and travels toward the light reception part 152 is received by the light reception part 152. In addition, also in the detection device 2 illustrated in FIGS. 25 and 26, the acceleration detection part 12 and the biological information detection part 15 are housed in the housing part 16. In this manner, the detection device 2 can be downsized in comparison with the case where the biological information detection part 15 and the acceleration detection part 12 are housed in separate housing parts. That is, the detection device 2 can reduce the amount of the stray light incident on the light reception part 152 while achieving size reception. In addition, the detection device 2 can reduce the amount of the stray light incident on the light reception part 152 in this manner, and therefore does not need to include a light blocking member for blocking light to prevent stray light incident on the light reception part 152. As a result, the detection device 1 can be more reliably downsized.

Note that in each of the second embodiment and the first to third modifications of the second embodiment described above, the shapes of the light emission part 151 and the light reception part 152 may be any shapes, and may be the same shape or different shapes. In addition, the second embodiment and the first to third modifications of the second embodiment described above may be combined with each other in any way. In addition, each of the second embodiment and the first to third modifications of the second embodiment described above may be combined with the first embodiment and the first to eighth modifications of the first embodiment in any way.

### Third Embodiment

A third embodiment is described below with reference to the accompanying drawings.

### Overview of Detection Device According To Third Embodiment

First, an overview of a detection device according to the third embodiment is described.

The detection device according to the third embodiment is attached to a predetermined portion of a subject person. In the detection device, the predetermined height direction is set as an upward direction. In addition, the detection device includes a base part, an acceleration detection part, a lid part, a control part, a light emission part, a light reception part, a first light blocking member, and a housing part. The base part includes a surface orthogonal to the height direction. The acceleration detection part is provided on the base part. The lid part covers the acceleration detection part together with the base part on the base part. The control part is provided on the lid part, and configured to detect the body movement based on the output of the acceleration detection part when the detection device is attached to the portion subject person. The biological information detection part includes a light emission part that emits light, and a light reception part that receives light. The first light blocking member is located between the biological information detection part and the control part in a width direction that intersects the height direction. The housing part houses the base part, the acceleration detection part, the lid part, the control part, the biological information detection part, and the first light blocking member. In this manner, the detection device can block a part of the light that is reflected on the control part in the housing part. As a result, the detection device can achieve size reduction while reducing incidence on the light reception part of stray light that is generated through irregular reflection in the housing part.

A configuration of the detection device according to the third embodiment is elaborated below. Note that in the third embodiment, the same components as those of the first embodiment are denoted with the same reference numerals, and the description thereof is omitted.

### Configuration of Detection Device According To Third Embodiment

A configuration of the detection device according to the third embodiment is described below with a detection device 3 as an example. In the third embodiment, viewing the detection device 3 toward a certain direction is referred to as viewing from that direction for convenience of description.

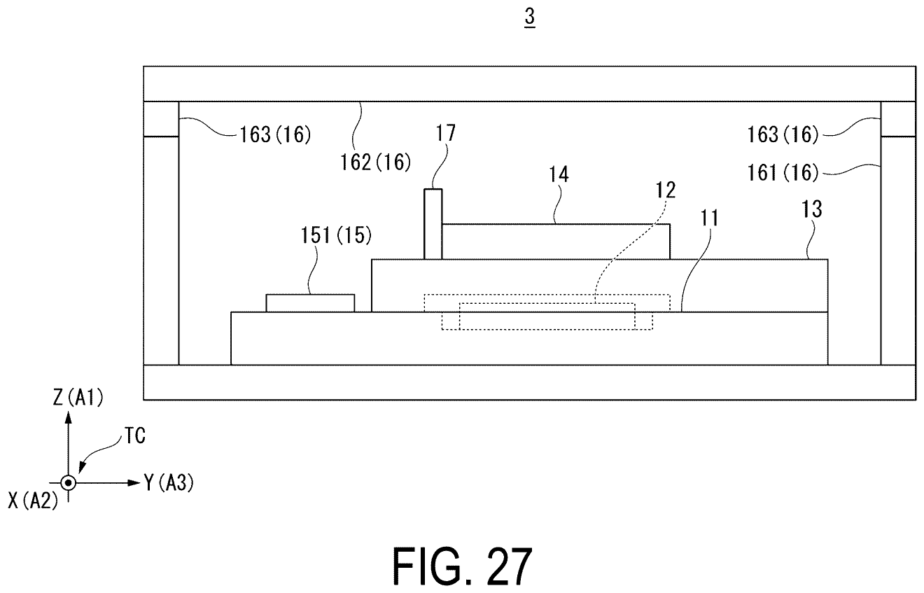

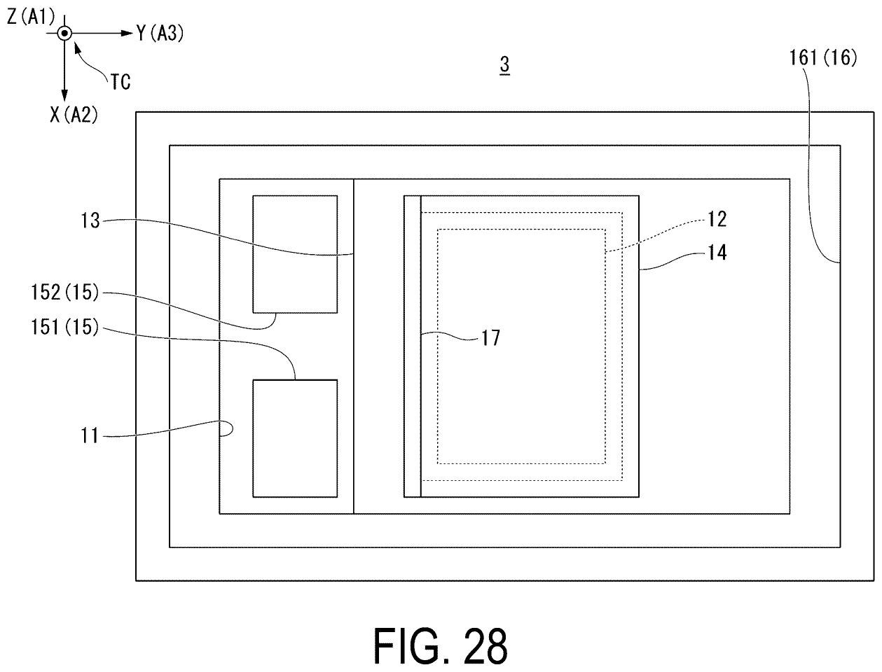

The detection device 3 has the same configuration as that of the detection device 1 except that a member as an example of the first light blocking member is provided. FIG. 27 is a front view illustrating an example of the configuration of the detection device 3. FIG. 28 is a top view of the detection device 3 illustrated in FIG. 27. Note that in FIG. 27, to clearly illustrate the inner configuration of the detection device 3, the member that covers the front surface of the inside of the detection device 3 is omitted. In addition, in FIG. 28, to clearly illustrate the inner configuration of the detection device 3, the member that covers the top surface of the inside of the detection device 3 is omitted.

In the example illustrated in FIGS. 27 and 28, the detection device 3 includes the base part 11, the acceleration detection part 12, the lid part 13, the control part 14, the biological information detection part 15, the housing part 16, and a first light blocking member 17.

The first light blocking member 17 is a member as an example of the above-described first light blocking member. The first light blocking member 17 may be any member as long as it can block light. The first light blocking member 17 is, but not limited to, a member made of black coated resin or opaque resin, for example. It should be noted that in this case, the first light blocking member 17 can efficiently absorb light. In the example illustrated in FIGS. 27 and 28, the first light blocking member 17 is a rectangular flat plate member with the long direction extending in the direction A2 and the short direction extending in the direction A1. The first light blocking member 17 is located between the biological information detection part 15 and the control part 14 in the direction A3. In this manner, the detection device 3 can block a part of the light that is reflected on the control part 14 in the housing part 16. This leads to suppression of irregular reflection of light in the housing part 16. As a result, the detection device 3 can reduce incidence on the light reception part 152 of stray light generated through irregular reflection of light in the housing part 16. In addition, the detection device 3 can reduce stray light from the control part 14 side to the light reception part 152 with the first light blocking member 17, and thus can increase the light reception range of the light reception part 152 for light that can be used for detection of biological information. Note that the first light blocking member 17 may be provided on the lid part 13 together with the control part 14 as illustrated in FIGS. 27 and 28, or may be supported by another supporting member so as to be located between the biological information detection part 15 and the control part 14 in the direction A3. It should be noted that the light reflection that causes the irregular reflection in the housing part 16 often occurs on the control part 14. The reason for this is that the lid part 13, which is a silicon cap, easily absorbs light, and that the base part 11, which is a glass substrate, easily transmit light. As such, it is desirable that the first light blocking member 17 be provided on the control part 14. The reason for this is that, in this case, the first light blocking member 17 can absorb the light reflected at the control part 14 before the light impinges on other members.

In addition, the first light blocking member 17 is in contact with the control part 14. In other words, the first light blocking member 17 is adjacent to the control part 14. In this manner, in the detection device 3, positioning using the first light blocking member 17 can be performed in the case where the control part 14 is provided on the lid part 13. This is useful because the assembly of the detection device 3 is facilitated.

In addition, in the example illustrated in FIGS. 27 and 28, the light emission part 151 is provided on the base part 11. As such, also in this example, the height of the top surface of the light emission part 151 is greater than the height of the bottom surface of the lid part 13. Therefore, also in this case, the detection device 3 can reduce the amount of light emitted from the light emission part 151 to the acceleration detection part 12. As a result, the detection device 3 can suppress the heating of the acceleration detection part 12 due to incidence of light. Note that the light emission part 151 may be configured to be provided on the base part 11 with a member such as a spacer therebetween.

In addition, also in the example illustrated in FIGS. 27 and 28, the light reception part 152 is provided on the base part 11 together with the light emission part 151. In addition, also in this example, the height of the top surface of the light reception part 152 is the same as the height of the top surface of the light emission part 151. Note that the height of the top surface of the light reception part 152 may be different from the height of the top surface of the light emission part 151. In FIG. 27, the light reception part 152 is hidden behind the light emission part 151. Note that the light reception part 152 may be provided on the lid part 13 with a member such as a spacer therebetween.

In addition, in the example illustrated in FIGS. 27 and 28, the light reception part 152 is provided side by side with the light emission part 151 in the direction A2 on the lid part 13.

In addition, in the example illustrated in FIGS. 27 and 28, the light reception part 152 is provided side by side with the control part 14 in the direction A3 on the lid part 13.

As described above, the detection device 3 illustrated in FIGS. 27 and 28 includes the first light blocking member 17. In this manner, the detection device 3 can block a part of the light that is reflected on the control part 14 in the housing part 16. This leads to suppression of irregular reflection of light in the housing part 16. As a result, the detection device 3 can reduce the light reception amount at the light reception part 152 of stray light generated through irregular reflection of light in the housing part 16. In addition, the detection device 3 can reduce stray light from the control part 14 side to the light reception part 152 with the first light blocking member 17, and thus can increase the light reception range of the light reception part 152 for light that can be used for detection of biological information. In addition, also in the detection device 3 illustrated in FIGS. 27 and 28, the acceleration detection part 12 and the biological information detection part 15 are housed in the housing part 16. In this manner, the detection device 3 can be downsized in comparison with the case where the biological information detection part 15 and the acceleration detection part 12 are housed in separate housing parts. Specifically, the detection device 3 can reduce incidence on the light reception part 152 of stray light generated through irregular reflection of light in the housing part 16 while reducing the size.

### First Modification of Third Embodiment

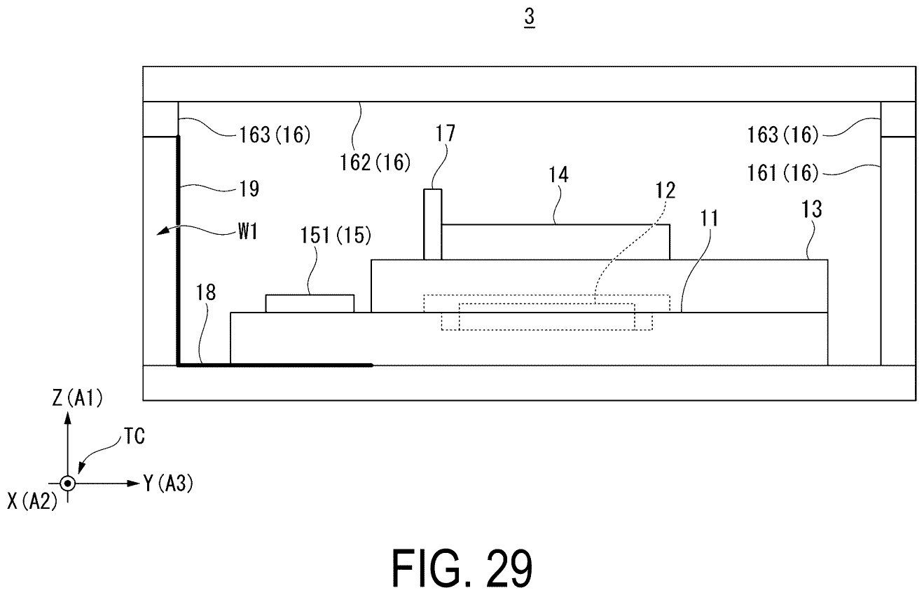

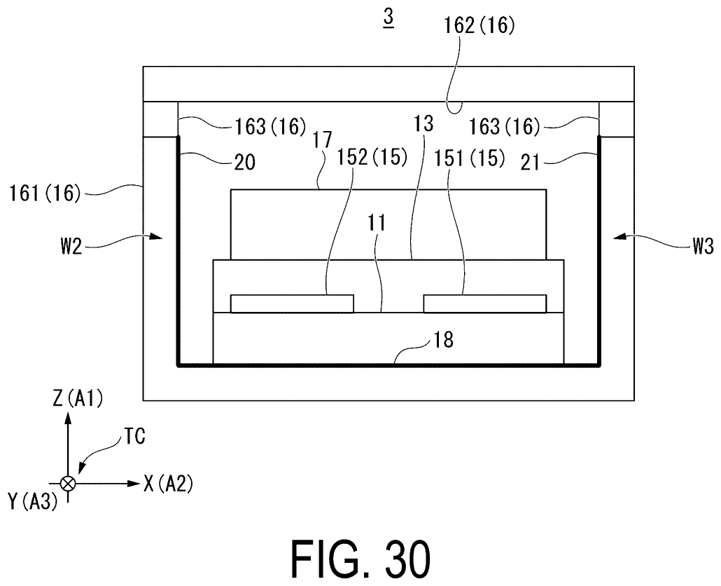

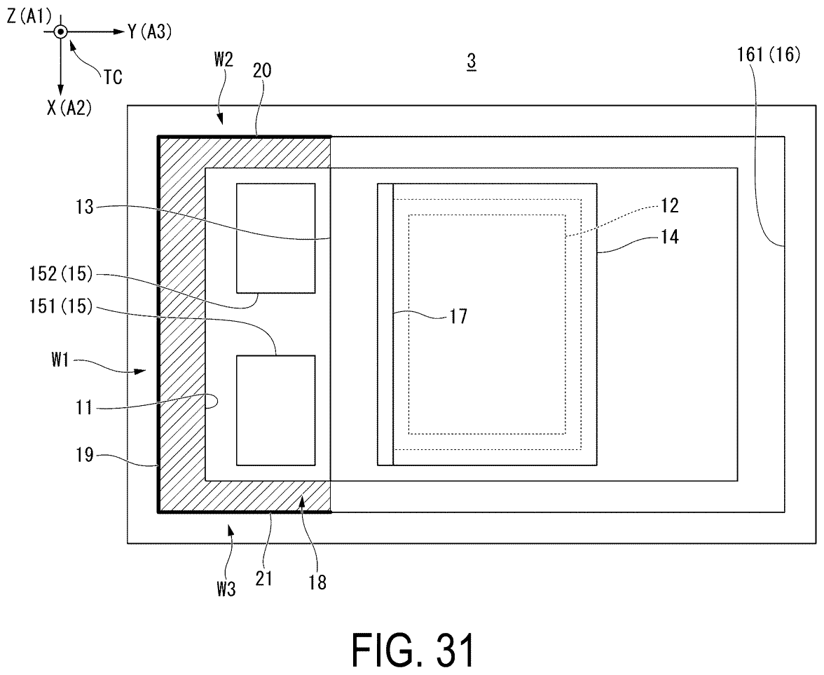

In a first modification of the third embodiment the detection device 3 includes, in addition to the configuration described in the third embodiment, a second light blocking member 18, a third light blocking member 19, a fourth light blocking member 20, and a fifth light blocking member 21. FIG. 29 is a front view illustrating the first modification of the configuration of the detection device 3. FIG. 30 is a side view of the detection device 3 illustrated in FIG. 29. FIG. 31 is a top view of the detection device 3 illustrated in FIG. 29. Note that in FIG. 29, to clearly illustrate the inner configuration of the detection device 3, the member that covers the front surface of the inside of the detection device 3 is omitted. In addition, in FIG. 30, to clearly illustrate the inner configuration of the detection device 3, the member that covers the side surface of the inside of the detection device 3 is omitted. In addition, in FIG. 31, to clearly illustrate the inner configuration of the detection device 3, the member that covers the top surface of the inside of the detection device 3 is omitted.

For convenience of description, in the following description, the inner wall on the negative direction side in the Y axis among the inner walls of the housing part 16 is referred to as first wall W1d, the inner wall on the negative direction side in the X axis among the inner walls of the housing part 16 is referred to as second wall W2d, and the inner wall on the positive direction side in the X axis among the inner walls of the housing part 16 is referred to as third wall W3. That is, the third wall W3 is the inner wall that faces the second wall W2 in the direction A2 among the inner walls of the housing part 16.

In the example illustrated in FIGS. 29 to 31, the second light blocking member 18 may be any member as long as it is a sheet-like member capable of blocking light, and is, but not limited to, a light shield sheet made of black coated resin or opaque resin, for example. It should be noted that in this case, the second light blocking member 18 can efficiently absorb light. The second light blocking member 18 is provided on the bottom surface and located between the bottom surface of the housing part 16 and the base part 11 in the direction A1. In this example, the second light blocking member 18 is a rectangular light shield sheet provided at the bottom surface and extended from the first wall W1 to the lid part 13 toward the direction A3 as viewed toward the direction opposite to the direction A1. In this manner, the second light blocking member 18 overlaps both the light emission part 151 and the light reception part 152 in the direction A1. In this manner, the second light blocking member 18 can absorb light not fully shielded by the lid part 13. In following description, the region on the bottom surface that overlaps the second light blocking member 18 in FIG. 31 is referred to as object region. The second light blocking member 18 may be configured to cover a part of the object region, or the entire object region as illustrated in FIGS. 29 to 31. In addition, the second light blocking member 18 may be configured to cover at least a part of the object region, as well as at least a part of the region other than the object region in the region on the bottom surface. In addition, the second light blocking member 18 may be configured to overlap one of the light emission part 151 and the light reception part 152, or may be configured to not overlap both the light emission part 151 and the light reception part 152.

The detection device 3 with the second light blocking member 18 can absorb at least a part of the light that impinges on the bottom surface of the housing part 16 such as light passing through the base part 11, with the second light blocking member 18. As a result, the detection device 3 can suppress irregular reflection of light in the housing part 16. That is, the detection device 3 can more reliably reduce the light reception amount at the light reception part 152 of stray light generated through irregular reflection of light in the housing part 16.

In addition, in the example illustrated in FIGS. 29 to 31, the third light blocking member 19 may be any member as long as it is a sheet-like member capable of blocking light, and is, but not limited to, a light shield sheet member made of black coated resin or opaque resin or the like, for example. It should be noted that in this case, the third light blocking member 19 can efficiently absorb light. The third light blocking member 19 is provided at the first wall W1. In this example, the third light blocking member 19 covers the entire first wall W1. However, the third light blocking member 19 may be configured to cover a part of the first wall W1.

The detection device 3 with the third light blocking member 19 can absorb at least a part of the light that impinges on the first wall W1 such as the light reflected at the metal of the surface of the acceleration detection part 12 as a silicon structure, with the third light blocking member 19. As a result, the detection device 3 can suppress irregular reflection of light in the housing part 16. That is, the detection device 3 can more reliably reduce incidence on the light reception part 152 of stray light generated through irregular reflection of light in the housing part 16.

In addition, in the example illustrated in FIGS. 29 to 31, the fourth light blocking member 20 may be any member as long as it is a sheet-like member capable of blocking light, and is, but not limited to, a light shield sheet member made of black coated resin or opaque resin or the like, for example. It should be noted that in this case, the fourth light blocking member 20 can efficiently absorb light. The fourth light blocking member 20 is provided at the second wall W2. In this example, the fourth light blocking member 20 covers the entire second wall W2. However, the fourth light blocking member 20 may be configured to cover a part of the second wall W2.

The detection device 3 with the fourth light blocking member 20 can absorb at least a part of the light that impinges on the second wall W2 such as light reflected at the metal of the surface of the acceleration detection part 12 as a silicon structure, with the fourth light blocking member 20. As a result, the detection device 3 can suppress irregular reflection of light in the housing part 16. That is, the detection device 3 can more reliably reduce incidence on the light reception part 152 of stray light generated through irregular reflection of light in the housing part 16.

In addition, in the example illustrated in FIGS. 29 to 31, the fifth light blocking member 21 may be any member as long as it is a sheet-like member capable of blocking light, and is, but not limited to, a light shield sheet member made of black coated resin or opaque resin or the like, for example. It should be noted that in this case, the fifth light blocking member 21 can efficiently absorb light. The fifth light blocking member 21 is provided at the third wall W3. In this example, the fifth light blocking member 21 covers the entire third wall W3. However, the fifth light blocking member 21 may be configured to cover a part of the third wall W3.

The detection device 3 with the fifth light blocking member 21 can absorb at least a part of the light that impinges on the third wall W3 such as the light reflected at the metal of the surface of the acceleration detection part 12 as a silicon structure, with the fifth light blocking member 21. As a result, the detection device 3 can suppress irregular reflection of light in the housing part 16. That is, the detection device 3 can more reliably reduce incidence on the light reception part 152 of stray light generated through irregular reflection of light in the housing part 16.

Note that the detection device 3 may not include some of the second light blocking member 18 to the fifth light blocking member 21. It should be noted that the greater the number of light blocking members of the four light blocking members, i.e., the second light blocking member 18 to the fifth light blocking member 21 in the detection device 3, the more the detection device 3 can absorb the light as the stray light in the housing part 16. Therefore, the detection device 3 desirably includes two or more of the second light blocking member 18 to the fifth light blocking member 21.

As described above, the detection device 3 illustrated in FIGS. 29 to 31 includes the second light blocking member 18 to the fifth light blocking member 21. In this manner, the detection device 3 can suppress irregular reflection of light in the housing part 16. Specifically, the detection device 3 can more reliably reduce incidence on the light reception part 152 of stray light generated through irregular reflection of light in the housing part 16.

### Second Modification of Third Embodiment

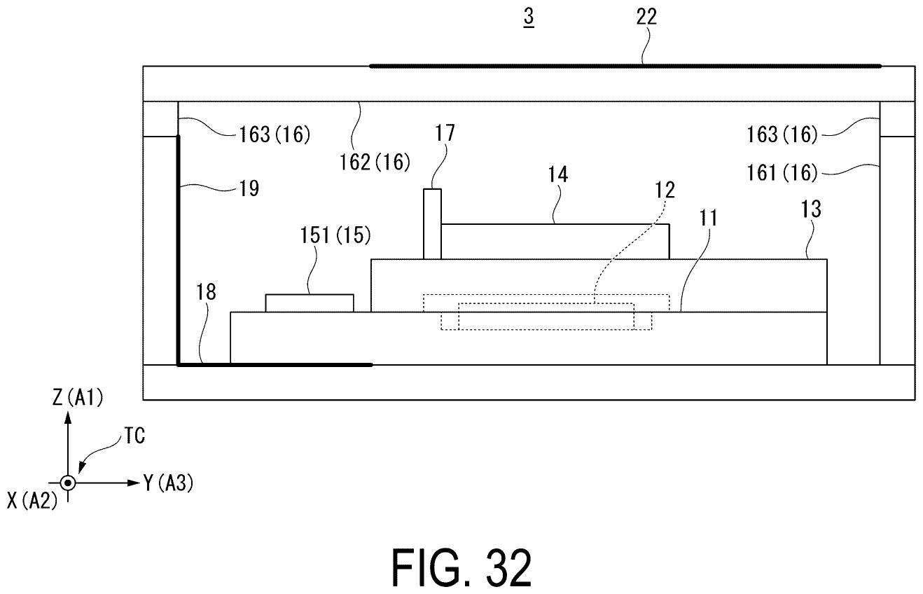

In a second modification of the third embodiment, the detection device 3 includes a sixth light blocking member 22 in addition to the configuration described in the first modification of the third embodiment. FIG. 32 is a front view illustrating a second modification of the configuration of the detection device 3. Note that in FIG. 32, to clearly illustrate the inner configuration of the detection device 3, the member that covers the front surface of the inside of the detection device 3 is omitted.

The sixth light blocking member 22 may be any member as long as it is a sheet-like member capable of blocking light, and is, but not limited to, a light shield sheet member made of black coated resin or opaque resin or the like, for example. It should be noted that in this case, the sixth light blocking member 22 can efficiently absorb light. The sixth light blocking member 22 is provided on the second member 162 so as to cover at least a part of the region that does not overlap the object region in the direction A1 in the region of the top surface of the second member 162. In this manner, the detection device 3 can allow for incidence on the housing part 16 of the light that is emitted from the light emission part 151 and reflected at the target portion, and can suppress incidence on the housing part 16 of the light such as ambient light other than this light. As a result, the detection device 3 can more reliably suppress irregular reflection of light in the housing part 16. Specifically, the detection device 3 can more reliably reduce incidence on the light reception part 152 of stray light generated through irregular reflection of light in the housing part 16.

As described above, the detection device 3 illustrated in FIG. 32 includes the sixth light blocking member 22. In this manner, the detection device 3 can suppress irregular reflection of light in the housing part 16. Specifically, the detection device 3 can more reliably reduce incidence on the light reception part 152 of stray light generated through irregular reflection of light in the housing part 16.

### Third Modification of Third Embodiment

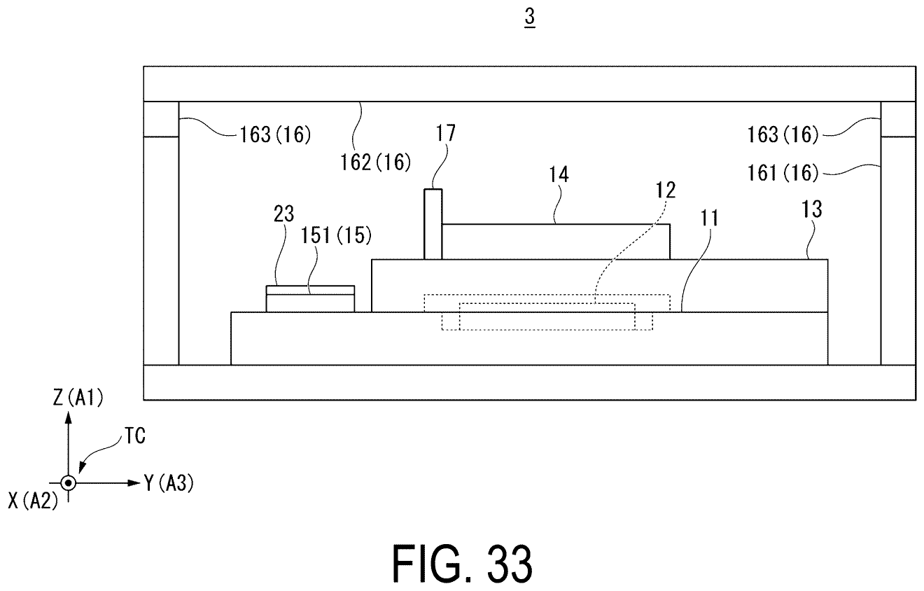

In a third modification of the third embodiment, the detection device 3 includes a filter 23 in addition to the configuration described in the third embodiment. FIG. 33 is a front view illustrating the third modification of the configuration of the detection device 3. Note that in FIG. 33, to clearly illustrate the inner configuration of the detection device 3, the member that covers the front surface of the inside of the detection device 3 is omitted.

The filter 23 is provided on the top surface of the light reception part 152. Note that in FIG. 33, the light reception part 152 is hidden behind the light emission part 151. The filter 23 is an angle limitation filter, for example. In the case where the filter 23 is an angle limitation filter, the detection device 3 can operate such that the light reception part 152 receives only incident light at a predetermined angle with respect to a virtual axis orthogonal to the top surface of the light reception part 152 as a central axis. The predetermined angle is 30°, for example. In this manner, the detection device 3 can cause the light reception part 152 to receive the light that is emitted from the light emission part 151 and reflected at the target portion, while suppressing a situation where the light other than this light such as ambient light is received at reception part 152. Note that the predetermined angle may be smaller than 30°, or greater than 30°. It should be noted that the greater the predetermined angle than 30°, the light is more easily received at the light reception part 152. As such, the predetermined angle is desirably equal to or smaller than 30°.

Note that the filter 23 may be an optical filter that transmits light in predetermined wavelength bands. In the case where the filter 23 is an optical filter, the detection device 3 can operate such that the light that is emitted from the light emission part 151 and reflected at the target portion is selectively received by the light reception part 152, and thus can suppress a situation where the light other than this light such as ambient light is received at reception part 152.

In addition, the filter 23 may be a filter composed of a stack of an angle limitation filter and an optical filter. In this manner, the detection device 3 can cause the light reception part 152 to receive the light that is emitted from the light emission part 151 and reflected at the target portion, while suppressing a situation where the light other than this light such as ambient light is received at reception part 152.

In addition, in the above-described detection device 3, a slit may be formed in the housing part 16 of the second member 162. In this case, this slit can achieve an effect similar to that of the case where the filter 23 is the angle limitation filter. Specifically, also in this case, the detection device 3 can cause the light reception part 152 to receive the light that is emitted from the light emission part 151 and reflected at the target portion, while suppressing a situation where the light other than this light such as ambient light is received at reception part 152.

In addition, the above-described detection device 3 may include a light shield wall that surrounds the periphery of the light reception part 152 around the direction A1, and extends from the light reception part 152 to the bottom surface of the second member 162 of the housing part 16. Also in this case, the detection device 3 can cause the light reception part 152 to receive the light that is emitted from the light emission part 151 and reflected at the target portion, while suppressing a situation where the light other than this light such as ambient light is received at reception part 152. Note that the height of the light shield wall may not reach the bottom surface of the second member 162.

Here, in each of the detection device 1, the detection device 2, and the detection device 3 described above, the biological information detection part 15 may include a plurality of light reception parts including the light reception part 152. It should be noted that as described above, each of the detection device 1, the detection device 2, and the detection device 3 can accurately detect the desired biological information by adjusting at least one of the wavelength band, intensity and the like of the light emitted from the light emission part 151. As such, the biological information detection part 15 may not include the plurality of light reception part. As such, in the above-described examples, the biological information detection part 15 include one light reception part 152. In addition, in each of the detection device 1, the detection device 2, and the detection device 3, the configuration in which the biological information detection part 15 does not include a plurality of light reception parts but includes one light reception part 152 is desirable because it leads to suppression of the increase in power consumption amount.

In addition, in each of the detection device 1, the detection device 2, and the detection device 3 described above, the control part 14 may be wrapped with a light blocking member. The reason for this is that the IC chip configured as the control part 14 can be malfunctioned by light. For the same reason, in the housing part 16 of the detection device 1, the detection device 2, and the detection device 3 described above, a light blocking member that blocks light incident on the control part 14 may be provided.

In addition, in each of the detection device 1, the detection device 2, and the detection device 3 described above, the biological information detection part 15 may include a plurality of light reception parts including the light reception part 152. It should be noted that as described above, each of the detection device 1, the detection device 2, and the detection device 3 can accurately detect the desired biological information by adjusting at least one of the wavelength band, intensity and the like of the light emitted from the light emission part 151. As such, the biological information detection part 15 may not include the plurality of light reception part. As such, in the above-described examples, the biological information detection part 15 include one light reception part 152. In addition, in each of the detection device 1, the detection device 2, and the detection device 3, the configuration in which the biological information detection part 15 does not include a plurality of light reception parts but includes one light reception part 152 is desirable because it leads to suppression of the increase in power consumption amount.

In addition, the matters described above may be appropriately combined.

### Supplementary Note 1

[1]

A detection device configured to be attached to a predetermined portion in a subject person, the detection device including a base part including a surface orthogonal to a first direction, the first direction being a predetermined direction set as an upper direction, an acceleration detection part provided on the base part, a lid part configured to cover the acceleration detection part together with the base part on the base part, a control part provided on the lid part and configured to detect a body movement of the subject person based on an output of the acceleration detection part when the detection device is attached to the portion, a light emission part configured to emit light, a light reception part configured to receive light, and a housing part configured to house the base part, the acceleration detection part, the lid part, the control part, the light emission part, and the light reception part. A height of a top surface of the light emission part in the first direction is equal to or greater than the height of the bottom surface of the lid part in the first direction.

[2]

The detection device according to [1], in which the control part detects biological information of the subject person in accordance with intensity of light received by the light reception part, and corrects the biological information in accordance with the detected body movement.

[3]

The detection device according to [1] or [2], in which both the light emission part and the light reception part are provided on the control part.

[4]

The detection device according to [1] or [2], in which both the light emission part and the light reception part are provided on the lid part or the base part.

[5]

The detection device according to [4], in which the height of the top surface of the light emission part in the first direction and a height of a top surface of the light reception part in the first direction are each equal to or smaller than a height of a top surface of the control part in the first direction.

[6]

The detection device according to [4], in which the control part extends toward a second direction that intersects the first direction, the light emission part is provided side by side with the control part in a third direction that intersects the first direction and the second direction, and the light reception part is provided side by side with the control part in the third direction, and provided side by side with the light emission part in the second direction.

[7]

The detection device according to [4], in which the control part extends toward a second direction that intersects the first direction, the light emission part is provided side by side with the control part in a third direction that intersects the first direction and the second direction, and in the third direction, the light reception part is provided side by side with the control part and the light emission part, and provided between the light emission part and the control part.

[8]

The detection device according to [4], in which the control part extends toward a second direction that intersects the first direction, the light emission part is provided side by side with the control part in a third direction that intersects the first direction and the second direction, and the light reception part is provided side by side with the control part in the second direction.

[9]

The detection device according to any one of [1] to [8], in which as viewed from the first direction, a size of the light emission part is different from a size of the light reception part.

The detection device according to [9], in which as viewed from the first direction, the light reception part is larger than the light emission part.

### Supplementary Note 2

[1]

A detection device configured to be attached to a predetermined portion in a subject person, the detection device including a base part including a surface orthogonal to a first direction, the first direction being a predetermined direction set as an upper direction, an acceleration detection part provided on the base part, a lid part configured to cover the acceleration detection part together with the base part on the base part, a control part provided on the lid part and configured to detect a body movement of the subject person based on an output of the acceleration detection part when the detection device is attached to the portion, a light emission part configured to emit light, a light reception part configured to receive light, and a housing part configured to house the base part, the acceleration detection part, the lid part, the control part, the light emission part, and the light reception part. A height of a top surface of the light emission part in the first direction is equal to or greater than the height of the bottom surface of the lid part in the first direction. The control part is provided between the light emission part and the light reception part in a direction in which the light emission part and the light reception part are disposed side by side.

[2]

The detection device according to [1], in which the control part detects biological information of the subject person in accordance with intensity of light received by the light reception part, and corrects the biological information in accordance with the detected body movement.

[3]

The detection device according to [1] or [2], in which both the light emission part and the light reception part are provided on the lid part or the base part.

[4]

The detection device according to [3], in which the height of the top surface of the light emission part in the first direction and a height of a top surface of the light reception part in the first direction are each equal to or smaller than a height of a top surface of the control part in the first direction.

[5]

The detection device according to [3], in which the control part extends toward the second direction that intersects the first direction, and the direction in which the light emission part and the light reception part are disposed side by side is a third direction that intersects the first direction and the second direction.

[6]

The detection device according to any one of [1] to [5], in which as viewed from the first direction, a size of the light emission part is different from a size of the light reception part.

[7]

The detection device according to [6], in which as viewed from the first direction, the light reception part is larger than the light emission part.

### Supplementary Note 3

[1]

A detection device configured to be attached to a predetermined portion in a subject person, the detection device including a base part including a surface orthogonal to a first direction, the first direction being a predetermined direction set as an upper direction, an acceleration detection part provided on the base part, a lid part configured to cover the acceleration detection part together with the base part on the base part, a control part provided on the lid part and configured to detect a body movement of the subject person based on an output of the acceleration detection part when the detection device is attached to the portion, a light emission part configured to emit light, a light reception part configured to receive light, and a housing part configured to house the base part, the acceleration detection part, the lid part, the control part, the light emission part, and the light reception part. A height of a top surface of the light emission part in the first direction is different from the height of the top surface of the light reception part in the first direction.

[2]

The detection device according to [1], in which the control part detects biological information of the subject person in accordance with intensity of light received by the light reception part, and corrects the biological information in accordance with the detected body movement.

[3]

The detection device according to [1] or [2], in which the control part extends toward a second direction that intersects the first direction, the light emission part is provided side by side with the control part in a third direction that intersects the first direction and the second direction, and the light reception part is provided side by side with the control part in the third direction, and provided side by side with the light emission part in the second direction.

[4] The detection device according to [1] or [2], in which the control part extends toward a second direction that intersects the first direction, the light emission part is provided side by side with the control part in a third direction that intersects the first direction and the second direction, and in the third direction, the light reception part is provided side by side with the control part and the light emission part, and provided between the light emission part and the control part.
[5]

The detection device according to [4], in which the control part is provided between the light emission part and the light reception part in the third direction.

[6]

The detection device according to any one of [1] to [5], in which the control part extends toward the second direction that intersects the first direction, and the direction in which the light emission part and the light reception part are disposed side by side is a third direction that intersects the first direction and the second direction.

[7]

The detection device according to [1] or [2], in which the light emission part is provided on any of the base part, the lid part and the control part, and the light reception part is provided on any of the two of the base part, the lid part and the control part where the light emission part is not provided.

[8]

The detection device according to any one of [1] to [6], in which the height of the top surface of the light reception part in the first direction is greater than the height of the top surface of the light emission part in the first direction.

[9]

The detection device according to any one of [1] to [6], in which the height of the top surface of the light emission part in the first direction is greater than the height of the top surface of the light reception part in the first direction.

The detection device according to any one of [1] to [9], in which as viewed from the first direction, a size of the light emission part is different from a size of the light reception part.

The detection device according to [10], in which as viewed from the first direction, the light reception part is larger than the light emission part.

### Supplementary Note 4

[1]

A detection device configured to be attached to a predetermined portion in a subject person, the detection device including a base part including a surface orthogonal to a height direction, the height direction being a predetermined direction set as an upper direction, an acceleration detection part provided on the base part, a lid part configured to cover the acceleration detection part together with the base part on the base part, a control part provided on the lid part and configured to detect a body movement of the subject person based on an output of the acceleration detection part when the detection device is attached to the portion, a biological information detection part including a light emission part that emits light and a light reception part that receives light, a first light blocking member located between the the biological information detection part and the control part in a width direction that intersects the height direction, and a housing part that houses the base part, the acceleration detection part, the lid part, the control part, the biological information detection part, and the first light blocking member.

[2]

The detection device according to [1], in which the control part detects biological information of the subject person in accordance with intensity of light received by the light reception part, and corrects the biological information in accordance with the detected body movement.

[3]

The detection device according to [1] or [2], in which the first light blocking member is provided on the lid part.

[4]

The detection device according to any one of [1] to [3], in which the light emission part and the light reception part are provided between the lid part and the first wall of the housing part in the width direction.

[5]

The detection device according to any one of [1] to [4], in which the light emission part and the light reception part are provided on the base part.

[6]

The detection device according to any one of [1] to [5], in which the first light blocking member is provided adjacent to the control part in the width direction.

[7]

The detection device according to any one of [1] to [6], further including a second light blocking member located between the bottom surface of the housing part and the base part in the height direction.

[8]

The detection device according to [7], in which in the height direction, the second light blocking member overlaps at least one of the light emission part and the light reception part.

[9]

The detection device according to [8], in which as viewed toward a direction opposite to the height direction, the second light blocking member extends from the first wall of the housing part to the lid part toward the width direction.

[10]

The detection device according to [9], further including a third light blocking member provided in the first wall.

[11]

The detection device according to [10], in which the housing part includes a second wall and a third wall, the second wall faces the third wall in the arrangement direction of the light reception part and the light emission part that intersects the height direction and the width direction, and the detection device includes a fourth light blocking member provided in the second wall and a fifth light blocking member provided in the third wall.

[12]

The detection device according to any one of [1] to [11], in which the light reception part is provided with an angle limitation filter.

[13]

The detection device according to any one of [1] to [12], in which the light reception part is provided with an optical filter.

[14]

The detection device according to any one of [1] to [13], in which the first light blocking member is opaque resin.

The embodiments of this disclosure have been described in detail with reference to the drawings. Specific configurations are not limited to this embodiment, but may be changed, replaced, deleted, etc., as long as they do not depart from the gist of this disclosure.

What is claimed is:

1. A detection device configured to be attached to a predetermined portion in a subject person, the detection device comprising:
a base part including a surface orthogonal to a first direction, the first direction being a predetermined direction set as an upper direction;
an acceleration detection part provided at the base part;
a lid part configured to cover the acceleration detection part together with the base part, at the base part;
a control part provided at the lid part and configured to detect a body movement of the subject person based on an output of the acceleration detection part when the detection device is attached to the portion;
a light emission part configured to emit light;
a light reception part configured to receive light; and
a housing part configured to house the base part, the acceleration detection part, the lid part, the control part, the light emission part, and the light reception part, wherein
a height of a top surface of the light emission part in the first direction is equal to or greater than the height of the bottom surface of the lid part in the first direction.

2. The detection device according to claim 1, wherein the control part detects biological information of the subject person in accordance with intensity of light received by the light reception part, and corrects the biological information in accordance with the detected body movement.

3. The detection device according to claim 1, wherein both the light emission part and the light reception part are provided at the control part.

4. The detection device according to claim 1, wherein both the light emission part and the light reception part are provided at the lid part or the base part.

5. The detection device according to claim 4, wherein the height of the top surface of the light emission part in the first direction and a height of a top surface of the light reception part in the first direction are each equal to or smaller than a height of a top surface of the control part in the first direction.

6. The detection device according to claim 4, wherein
the control part extends toward a second direction that intersects the first direction,
the light emission part is provided side by side with the control part in a third direction that intersects the first direction and the second direction, and
the light reception part is provided side by side with the control part in the third direction, and provided side by side with the light emission part in the second direction.

7. The detection device according to claim 4, wherein
the control part extends toward a second direction that intersects the first direction,
the light emission part is provided side by side with the control part in a third direction that intersects the first direction and the second direction, and
in the third direction, the light reception part is provided side by side with the control part and the light emission part, and provided between the light emission part and the control part.

8. The detection device according to claim 4, wherein
the control part extends toward a second direction that intersects the first direction,
the light emission part is provided side by side with the control part in a third direction that intersects the first direction and the second direction, and
the light reception part is provided side by side with the control part in the second direction.

9. The detection device according to claim 1, wherein as viewed from the first direction, a size of the light emission part is different from a size of the light reception part.

10. The detection device according to claim 9, wherein as viewed from the first direction, the light reception part is larger than the light emission part.
